(19) United States
(12) Patent Application Publication
(10) Pub. No.: US 2004/0161813 A1
(43) Pub. Date: Aug. 19, 2004

(54) Structure of beta-ketoacyl-[acyl carrier protein] synthases complexed with inhibitors and methods of use thereof

(75) Inventors: Charles O. Rock, Bartlett, TN (US); Allen Price, Memphis, TN (US); Stephen White, Memphis, TN (US)

Correspondence Address: BUTLER, SNOW, O'MARA, STEVENS & CANNADA PLLC, 6075 POPLAR AVENUE, SUITE 500, MEMPHIS, TN, 38119, US

(73) Assignee: St. Jude Children's Research Hospital, Memphis, TN (US)

(21) Appl. No.: 09/917,331

(22) Filed: Jul. 27, 2001

Related U.S. Application Data
(60) Provisional application No. 60/223,222, filed on Aug. 4, 2000.

(51) Int. Cl. C12Q 1/48

(52) U.S. Cl. 435/15; 435/193; 700/90; 702/27

(57) ABSTRACT

Crystals of FabB-inhibitor complexes are disclosed that allow the three-dimensional structure of the FabB-inhibitor complex to be determined. The use of this structural information to identify and/or design drugs to treat bacterial infections is also disclosed.

### CROSS-REFERENCE TO RELATED APPLICATIONS

The present application is a non-provisional application claiming the priority of copending provisional U.S. Ser. No. 60/223,222 filed Aug. 4, 2000, the disclosure of which is hereby incorporated by reference in its entirety. Applicants claim the benefits of this application under 35 U.S.C. § 119(e).

### RESEARCH SUPPORT

The research leading to the present invention was supported in part by the National Institutes of Health Grants GM34496, GM44973, the Cancer Center Support (CORE) grant CA-21765. The government may have certain rights in the present invention.

Support for this invention was also provided by the AMERICAN LEBANESE SYRIAN ASSOCIATED CHARITIES.

### REFERENCE TO TABLE SUBMITTED ON COMPACT DISC

Two compact discs are included with the instant filing. The compact discs contain identical material. The material on the compact discs is hereby incorporated by reference in its entirety in accordance with 37 CFR § 1.77(b)(4). The compact discs each contain two files, with both files being dated Jul. 19, 2001. The files (i) are labeled as Table III and Table IV, (ii) are in Microsoft Word and (iii) contain 2337 KB and 2452 KB respectively. The compact discs contain the atomic coordinates for the FabB-cerulenin complex (Table III) and the atomic coordinates for the FabB-TLM complex (Table IV), as further described in the text below. A hard copy of the atomic coordinates is also included in the Appendix that follows the Sequence Listing. The hard copy of the atomic coordinates for the FabB-cerulenin complex is denoted as Table III, whereas that of the atomic coordinates for the FabB-TLM complex is denoted as Table IV.

The present invention provides crystals of β-ketoacyl-[acyl carrier protein] synthases complexed with inhibitors. The three-dimensional structural information is included in the invention. The present invention provides procedures for identifying agents that can inhibit bacterial cell growth through the use of rational drug design predicated on the crystallographic data.

### BACKGROUND OF THE INVENTION

Drug resistance in infectious organisms has become a serious medical problem, and fatty acid synthesis has emerged as a promising target for the development of novel therapeutic agents. Lipid synthesis is not only essential to cell viability, but specificity for bacteria and other infectious organisms can be achieved by taking advantage of the organizational and structural differences that exist in the fatty acid synthetic systems of different organisms. There are two major types. The associated, or type I, systems exist in higher organisms such as mammals, and comprise a single, multifunctional polypeptide (1). The dissociated, or type II, fatty acid synthases exist in bacteria and plants, and are composed of a collection of discrete enzymes that each carry out an individual step in the cycles of chain elongation (2,3). Triclosan (4,5) and isoniazid (6) are two commonly used antibacterial agents that target fatty acid synthesis.

The type II system has been most extensively studied in Escherichia coli where the three β-ketoacyl-acyl carrier protein synthases (β-ketoacyl-ACP synthases) have emerged as important regulators of the initiation and elongation steps in the pathway. These enzymes catalyze the Claisen condensation reaction, transferring an acyl primer to malonyl-ACP, thereby creating a β-ketoacyl-ACP that has been lengthened by two carbon units. Two of these synthases are elongation condensing enzymes. Synthase I (FabB) is required for a critical step in the elongation of unsaturated fatty acids. Mutants (fabB) lacking synthase I activity require supplementation with exogenous unsaturated fatty acids to support growth (7,8). Synthase II (FabF) controls the temperature-dependent regulation of fatty acid composition (9,10). Mutants lacking synthase II (fabF) are deficient in the elongation of palmitoleate to cis-vaccenate, but grow normally under standard culture conditions (9,11,12). The third synthase functions as the initiation condensing enzyme. Synthase III (FabH) catalyzes the first condensation step in the pathway and is thus ideally situated to govern the rate of fatty acid synthesis (13-16). Unlike FabB and FabF, FabH enzymes use an acyl-CoA rather than an acyl-ACP as the primer (15-18). FabH is further distinguished by a His-Asn-Cys (19,20) catalytic triad in contrast to the His-His-Cys triad in the FabB (21) and FabF (22) enzymes.

The crystal structures of all three condensing enzymes from E. coli (FabB, FabF and FabH) have now been determined (19-22). Their primary structures are clearly related, and these translate into similar dimeric structures and active site architectures. The structures of the monomers comprise an internally duplicated helix-sheet-helix motif, and the active site is located at the convergence of the pseudo dyad-related α-helices at the center of the molecule. The buried active site is accessed by a tunnel that accommodates the 4′-phosphopantetheine prosthetic group of ACP (and also CoA in the case of FabH). The active site is functionally and architecturally divided into halves, and each half is associated with one of the duplicated motifs. The initial transacylation half-reaction, that attaches the acyl primer to the active site cysteine, is facilitated by an α-helix dipole and an oxyanion hole. The decarboxylation half-reaction that transfers the acyl primer to malonyl-ACP is accelerated by the formation of two adjacent hydrogen bonds to the thioester carbonyl of the incoming malonyl-ACP. The hydrogen bond donors are two histidines in the FabB/FabF class and a histidine and an asparagine in the FabH class. In addition, the side chain of a conserved phenylalanine promotes the decarboxylation step in both types of enzymes. This scheme is supported by mutagenesis studies of FabH (20) and differs somewhat from the mechanisms proposed by others (19,22).

Two natural products inhibit type II fatty acid synthesis by blocking the activity of one or more of the β-ketoacyl-ACP synthases. Cerulenin is an irreversible inhibitor of β-ketoacyl-ACP synthases I and II (23-25) and forms a covalent adduct with the active site cysteine (26). Cerulenin is not a selective antibacterial because it is also a potent inhibitor of the condensation reaction catalyzed by the mammalian multifunctional (type I) fatty acid synthase (27,28). However, cerulenin and related compounds have antineoplastic activity (29) and reduce food intake and body weight in mice (30).

Thiolactomycin (TLM) is a unique thiolactone molecule that reversibly inhibits type II, but not type I, fatty acid synthases (31,32), and is effective against many pathogens. The antibiotic is not toxic to mice and affords significant protection against urinary tract and intraperitoneal bacterial infections (33). TLM is active against gram-negative anaerobes associated with periodontal disease (34) and exhibits antimycobacterial action by virtue of its inhibition of mycolic acid synthesis (35). TLM also has activity against malaria (36) and trypanosomes (37), extending the potential for using this template as a platform to develop more antimicrobials.

Bacterial infections remain among the most common and deadly causes of human disease. For example, Streptococci are known to cause otitis media, conjunctivitis, pneumonia, bacteremia, meningitis, sinusitis, pleural empyema and endocarditis. In addition, virulent strains of E. coli can cause severe diarrhea, a condition that kills a million more people (3 million) worldwide every year than malaria [D. Leff, BIOWORLD TODAY, 9:1,3 (1998)]. Indeed, infectious diseases are the third leading cause of death in the United States and the leading cause of death worldwide [Binder et al., Science 284:1311-1313 (1999)].

Although, there was initial optimism in the middle of the 20th century that diseases caused by bacteria would be quickly eradicated, it has become evident that the so-called “miracle drugs” are not sufficient to accomplish this task. Indeed, antibiotic resistant pathogenic strains of bacteria have become commonplace, and bacterial resistance to the new variations of these drugs appears to be outpacing the ability of scientists to develop effective chemical analogs of the existing drugs [See, Stuart B. Levy, The Challenge of Antibiotic Resistance, in Scientific American, 46-53 (March, 1998)]. Therefore, new approaches to drug development are necessary to combat the ever-increasing number of antibiotic-resistant pathogens.

Classical penicillin-type antibiotics effect a single class of proteins known as autolysins. Therefore, the development of new drugs which effect an alternative bacterial target protein would be desirable. Such a protein target ideally would be indispensable for bacterial survival.

Therefore, there is a need to develop methods for identifying drugs that interfere with β-ketoacyl-ACP synthases. A superior method for drug screening relies on structure based rational drug design. In such cases, a three dimensional structure of the protein complexed with an inhibitor is determined and potential agonists and/or antagonists are designed with the aid of computer modeling [Bugg et al., Scientific American, Dec.: 92-98 (1993); West et al., TIPS, 16:67-74 (1995); Dunbrack et al., Folding & Design, 2:27-42 (1997)].

Therefore, there is a need for obtaining crystals of bacterial enzymes such as β-ketoacyl-ACP synthases that are complexed with inhibitors that are amenable to high resolution X-ray crystallographic analysis. In addition, there is a need for determining the three-dimensional structure of such complexes. Furthermore, there is a need for developing procedures of structure based rational drug design using such three-dimensional information. Finally, there is a need to employ such procedures to develop new drugs against bacteria and other infectious organisms.

The citation of any reference herein should not be construed as an admission that such reference is available as “Prior Art” to the instant application.

### SUMMARY OF THE INVENTION

The present invention provides a crystal of a binding complex between FabB and thiolactomycin that effectively diffracts X-rays for the determination of the atomic coordinates to a resolution of better than 3.5 Angstroms. In a preferred embodiment the FabB is E. coli FabB. In a particular embodiment the crystal has the space group of P2₁2₁2₁ and a unit cell of dimensions of a=59.1 b=139 and c=211.9 Angstroms.

The present invention further provides a crystal of a binding complex between FabB and cerulenin that effectively diffracts X-rays for the determination of the atomic coordinates to a resolution of better than 3.5 Angstroms. In a preferred embodiment the FabB is E. coli FabB. In a particular embodiment the crystal has the space group of P2₁2₁2₁ and a unit cell of dimensions of a=59.2 b=139.6 and c=212.2 Angstroms.

The present invention also provides a method of obtaining a crystal of an inhibitor-FabB complex comprising growing a crystal of the inhibitor-FabB complex in a buffered solution containing 2.0 M ammonium sulfate, and 20% PEG 400. In a particular embodiment the crystal is grown using batch crystallization. In another embodiment, the crystal is grown using vapor diffusion. In yet another embodiment, the crystal is grown using microdialysis.

The present invention further provides an apparatus that comprises a representation of an FabB-inhibitor binding complex. In one such embodiment the FabB-inhibitor binding complex is an FabB-cerulenin binding complex. In another such embodiment the FabB-inhibitor binding complex is the FabB-TLM binding complex. Preferably the FabB is an E. coli FabB having the amino acid sequence of SEQ ID NO:2. One such apparatus is a computer that comprises the FabB-inhibitor binding complex in computer memory. In a particular embodiment, the computer comprises a machine-readable data storage medium which contains data storage material that is encoded with machine-readable data which comprises the atomic coordinates obtained from a crystal of an FabB-inhibitor binding complex, e.g., the FabB-cerulenin binding complex or the FabB-TLM binding complex. Preferably the computer comprises a machine-readable data storage medium which contains data storage material that is encoded with machine-readable data which comprises the atomic coordinates of Table III and/or Table IV. In a particular embodiment, the computer comprises a machine-readable data storage medium which contains data storage material that is encoded with machine-readable data which comprises the structural (i.e., atomic) coordinates obtained from a crystal of the FabB-inhibitor binding complex. Preferably the computer further comprises a working memory for storing instructions for processing the machine-readable data, a central processing unit coupled to both the working memory and to the machine-readable data storage medium for processing the machine readable data into a three-dimensional representation of the FabB-inhibitor binding complex. In a preferred embodiment, the computer also comprises a display that is coupled to the central-processing unit for displaying the three-dimensional representation.

Thus the present invention provides a computer comprising the atomic coordinates of Table III in an electronic or magnetic medium as well as a computer comprising the atomic coordinates of Table IV in an electronic or magnetic medium.

Another aspect of the present invention is a method of identifying an agent for use as an inhibitor of bacterial fatty acid synthesis. One such embodiment uses a crystal of a binding complex between FabB and cerulenin of the present invention. Another such embodiment uses a crystal of a binding complex between FabB and thiolactomycin. One such method comprises selecting a potential agent by performing rational drug design with the three-dimensional coordinates (i.e., the atomic coordinates) determined from the crystal. Preferably the selecting is performed in conjunction with computer modeling. The potential agent is then contacted with a β-ketoacyl-ACP synthase and the activity of the β-ketoacyl-ACP synthase is determined (e.g., measured). A potential agent is identified as an agent that inhibits bacterial fatty acid synthesis when there is a decrease in the activity of the β-ketoacyl-ACP synthase.

In a preferred embodiment the method further comprises growing a supplemental crystal containing FabB formed in the presence of the potential agent. The crystal effectively diffracts X-rays for the determination of the atomic coordinates to a resolution of better than 5.0 Angstroms. The three-dimensional coordinates (i.e., the atomic coordinates) of the supplemental crystal are then determined with molecular replacement analysis and a second generation agent is selected by performing rational drug design with the three-dimensional coordinates determined for the supplemental crystal. The selecting is preferably performed in conjunction with computer modeling.

The present invention also provides methods of identifying agents that inhibit bacterial growth using the atomic coordinates obtained from a crystal of the present invention. One such method comprises selecting a potential agent by performing rational drug design with the three-dimensional coordinates determined for the crystal. The selecting is preferably performed in conjunction with computer modeling. The potential agent is contacted with a bacterial culture and then the growth of the bacterial culture is determined (e.g., measured). A potential agent is identified as an agent that inhibits bacterial growth when there is a decrease in the growth of the bacterial culture.

In a preferred embodiment the method further comprises growing a supplemental crystal containing FabB formed in the presence of the potential agent. The crystal effectively diffracts X-rays for the determination of the atomic coordinates to a resolution of better than 5.0 Angstroms, and then determining the three-dimensional coordinates of the supplemental crystal with molecular replacement analysis. Finally a second generation agent is selected by performing rational drug design with the three-dimensional coordinates determined for the supplemental crystal. Preferably the selecting is performed in conjunction with computer modeling.

The method of can further comprise contacting the second generation agent with a eukaryotic cell and then measuring the amount of proliferation of the eukaryotic cell. A potential agent is identified as an agent for inhibiting bacterial growth when there is no change in the proliferation of the eukaryotic cell.

In addition, the atomic coordinates in Table III and/or IV can be used directly in the above assays.

These and other aspects of the present invention will be better appreciated by reference to the following drawings and Detailed Description.

### BRIEF DESCRIPTION OF THE DRAWINGS

FIGS. 1A-1B show the inhibition of E. coli condensing enzymes by TLM and cerulenin. FIG. 1A is the inhibition of FabF (æ), FabB (∘) and FabH (▪) by TLM. The IC₅₀ values were: FabF, 6 μM; FabB, 25 μM; and FabH, 110 μM. FIG. 1B is the inhibition of FabF (●), FabB (∘) and FabH (▪) by cerulenin. The IC₅₀ values were: FabF, 20 μM; FabB, 3 μM; and FabH, >700 μM. The activities of the three condensing enzymes were compared using the radiochemical assays described under “Experimental Procedures.”

FIG. 2 shows that FabB is reversibly inhibited by TLM and irreversibly inhibited by cerulenin. The time course for the inhibition of FabB activity under five experimental conditions was determined to address the reversibility of the antibiotics. Time course for the FabB reaction in the absence of inhibitors (▾), in the presence of 20 μM TLM (●) or 3 μM cerulenin (∘). FabB was preincubated with either 20 μM TLM (▪) or 3 μM cerulenin (□) for 30 min prior to initiation of the reaction by the addition of the other substrates. FabB activity was measured using the radiochemical assay with myristoyl-ACP as the substrate as described under “Experimental Procedures” in the Example below.

FIGS. 3A-3B show the structure of the FabB-TLM binary complex. FIG. 3A is the stereo diagram of the complex. TLM is shown with magenta bonds. Secondary structural elements are shown in orange, as are all alpha carbons. The atoms in TLM, the atoms in the active site residues (H298, H333, C163, F392), and important protein backbone atoms are color coded: carbon, black; nitrogen, blue; oxygen, red; and sulfur, yellow. Hydrophobic residues are shown green. Water molecules are shown light blue. Hydrogen bonds are shown as dotted red lines. The native, unbound conformations of the active site residues are shown in purple. TLM forms hydrogen bonds with the two active site histidines, H298 and H333, and to a network of waters which is held in place by the carbonyl oxygen of V270 and by the amine group of G305. TLM is further stabilized by the intercalation of its isoprenoid tail into the space between P272 and its associated peptide bond and the peptide bond between G391 and F392. FIG. 3B shows the electron density of bound TLM. The electron density is contoured at the one sigma level.

FIGS. 4A-4B show the structure of the FabB-cerulenin covalent complex. FIG. 4A shows the stereo diagram of the complex. Cerulenin is shown with yellow bonds. The rest of the coloring scheme in this figure is the same as in FIG. 3. Cerulenin forms a covalent bond with the active site cysteine (C163). Its O2 oxygen forms hydrogen bonds with the two active site histidines, H298 and H333. The O3 oxygen sits in the oxyanion hole, forming hydrogen bonds to the amide of F392 and the amide of C163. The tail of cerulenin occupies a long hydrophobic cavity, which normally contains the growing acyl chain of the natural substrate. FIG. 4B shows the electron density of bound cerulenin. The electron density is contoured at the one sigma level.

FIGS. 5A-5C show that FabB[H333N] is resistant to TLM. FIG. 5A depicts the specific acitivities of FabB and FabB[H333N]. The specific activity of FabB (∘) was 425±6 pmoles/min/μg whereas FabB[H333N] (●) had 1.6% of the condensation activity of the wild-type protein (6.6±0.4 pmoles/min/μg). FIG. 5B shows a comparison of the inhibition of FabB[H333N] (●) and FabB (∘) by TLM. The IC₅₀ for FabB was 25 μM compared to the higher IC₅₀ of FabB[H333N] (350 EM). FIG. 5C shows a comparison of the inhibition of FabB[H333N] (●) and FabB (∘) by cerulenin. The IC₅₀ for FabB was 3 μM compared to the higher IC₅₀ of FabB[H333N] (30 μM). The assays were performed using 14:0-ACP as the primer for the radiochemical assay described under “Experimental Procedures” in the example below.

FIG. 6 shows an overlay of TLM and cerulenin in the FabB active site. The FabB-TLM and FabB-cerulenin structures were superimposed to illustrate the differences in the binding of the antibiotics in the active site. The coloring scheme in this figure is the same as in FIGS. 3 and 4. TLM binds on the malonyl-ACP side and cerulenin occupies the acyl-enzyme intermediate half. The O1 of TLM and O2 of cerulenin are the only portions of the antibiotics that overlap in the structure and they form hydrogen bonds with the His-His dyad in the active site. Note that the protein structure shown is that of the FabB-TLM complex. Binding of the two antibiotics results in essentially identical changes in the conformations of the active site residues.

FIGS. 7A-7B show schematic diagrams illustrating how cerulenin and TLM mimic substrates in the active site of FabB. FIG. 7A shows how cerulenin mimics the condensation transition state and spans the two halves of the active site. The thiolactone ring of TLM mimics the bent conformation of the thiomalonate, and this is emphasized by the shaded atoms. The O1 oxygens form hydrogen bonds with His298 and His333 and the C1, C2 and C3 carbons of malonate are mimicked by the C1, C2 and C9 carbons of TLM. The O2 of TLM points out the active site tunnel which would be occupied by the pantetheine arm of the malonyl-ACP substrate. FIG. 7B shows the thiolactone ring of TLM mimics the bent conformation of the thiomalonate, and this is emphasized by the shaded atoms. Cerulenin mimics the condensation transition state and spans the two halves of the active site. The O3 of cerulenin lies in the oxyanion hole formed by the amides of Cys163 and Phe392 enclosed by the phenyl sidechain of Phe392. This structure mimics the postulated location of the oxyanion of the tetrahedral transition state. The sidechain of Cys163 rotates in the cerulenin structure to form a covalent bond with C2, but in the transition state, it is postulated to reside in the location observed in the native enzyme. The acyl chain of cerulenin feeds into the hydrophobic groove that accommodates the long-chain acyl-enzyme intermediate.

FIG. 8 depicts a schematic of a computer comprising a central processing unit (“CPU”), a working memory, a mass storage memory, a display terminal, and a keyboard that are interconnected by a conventional bidirectional system bus. The computer can be used to display and manipulate the structural data of the present invention.

### DETAILED DESCRIPTION OF THE INVENTION

The present invention discloses the structures of the FabB-TLM and FabB-cerulenin complexes, and identifies structural features that define the differences in the biochemical mode of action and target selectivity of the two antibiotics. Furthermore, the present invention has validated the understanding of the mechanisms of antibiotic binding through the mutagenesis of a key residue involved in the protein-drug interaction, and the subsequent assay of the mutant. This work contributes not only to the development of new antibacterials that target the condensation step in type II fatty acid synthesis, but also to the understanding of the condensation reaction mechanism.

The β-ketoacyl-acyl carrier protein synthases (β-ketoacyl-ACP synthases) are key regulators of type II fatty acid synthesis and are the targets for two natural products, thiolactomycin (TLM) and cerulenin. Biochemical analysis of the three purified condensing enzymes revealed that synthase II (FabF) was the most sensitive to TLM, whereas FabB was the most sensitive to cerulenin. The high-resolution structures of the FabB-TLM and FabB-cerulenin binary complexes are disclosed herein (see Tables III and IV). TLM mimics malonyl-ACP in the FabB active site. It forms strong hydrogen bond interactions with the two catalytic histidines, and the unsaturated alkyl side chain interaction with a small hydrophobic pocket is stabilized by π stacking interactions. Cerulenin binding mimics the condensation transition state. The subtle differences between the FabB-cerulenin and FabF-cerulenin structures explain the differences in the sensitivity of the two enzymes to the antibiotic and may reflect the distinct substrate specificities that differentiate the two enzymes.

The FabB[H333N] protein was prepared to convert the FabB His-His-Cys active site triad into the FabH His-Asn-Cys configuration to test the importance of the two His residues in TLM and cerulenin binding. [Since SEQ ID NO:1 contains amino acid residues 2 to 404 of the E.coli FabB (SEQ ID NO:2), H333 corresponds to amino acid residue 332 of SEQ ID NO:1, but 333 of SEQ ID NO:2]. FabB[H333N] was significantly more resistant to both antibiotics than FabB, illustrating that the two-histidine active site architecture is critical to high affinity binding. These data provide a structural framework for understanding antibiotic sensitivity within this group of enzymes.

### Conservation of Bacterial β-ketoacyl ACP Synthases

While the pathway for the biosynthesis of fatty acids is similar in prokaryotes and eukaryotes, the organization of the biosynthetic apparatus is very different. Vertebrates and yeast possess a fatty acid synthase (FAS) in which all of the enzymatic activities are encoded on one or two polypeptide chains, respectively, and the acyl carrier protein (ACP) is an integral part of the complex. In contrast, in bacterial FAS each of the reactions is catalyzed by distinct monofunctional enzymes and the ACP is a discrete protein (below). There is therefore considerable potential for selective inhibition of the bacterial systems and thus, an excellent opportunity to identify novel drugs against pathogens that cause massive human suffering and mortality such as tuberculosis, as well as multi-antibiotic resistant organisms which require specific targeting.

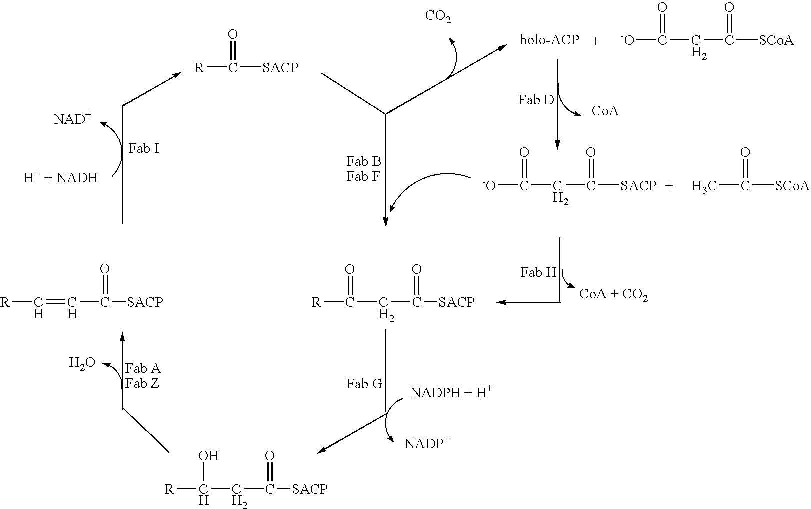

### THE TYPE II FATTY ACID BIOSYNTHETIC APPARATUS IN PROKARYOTES

Most bacteria possess three β-ketoacyl-ACP synthases (KAS) that are central to the initiation and elongation steps in de novo fatty acid synthesis and play a pivotal role in regulation of the entire pathway. They have discrete functions and preferred substrates for extension as well as distinct inhibition profiles as illustrated by the fact that FabF, FabB and FabH are the targets of TLM, whilst cerulenin inhibits only FabB and FabH.

There are basically two groups of condensing enzymes found in type II fatty acid biosynthesis (1) (Table 1). One group (Panel A) catalyzes the elongation reactions in the cycle to extend the acyl chain. These enzymes use acyl-ACP as the primer. There are two types of condensing enzymes in this group called FabB and FabF. Whether one or both of the enzymes occurs in a particular organism depends on the fatty acid composition produced by the pathway. Unsaturated fatty acid biosynthesis requires a FabB-type enzyme, whereas saturated fatty acid synthesis requires the FabF-class of enzymes. There are two condensing enzyme in M tuberculosis which have been termed KasA and KasB. Whereas these enzymes clearly belong to the Panel A group of condensing enzymes, it is not clear as to whether they are both more closely related to FabF or FabB. A signature His-His-Cys catalytic triad characterizes the Panel A enzyme active sites. The second group of condensing enzyme (Panel B) catalyzes the initial condensation reaction in the pathway using an acyl-CoA as the primer. In E. coli, S. pneumoniae and H. influenza the FabH enzymes use acetyl-CoA, as the primer and synthesize straight-chain fatty acids. In S. aureus, the FabH uses branched-chain acyl-CoA primers and produces and in M. tuberculosis FabH uses long-chain acyl-CoA to prime the type II system that produces the cell wall mycolic acids. The FabH (Panel B) enzymes are distinguished from the FabB/F (Panel A) enzymes in that they have a His-Asn-Cys catalytic triad at the active site.

TABLE I
Occurrence of condensing enzymes in bacteria.

| | Panel A | Panel B |
|---|---|---|
| Organism | (FabB/F enzymes) | (FabH enzymes) |
| Escherichia coli | eFabB, eFabF | eFabH |
| Mycobacterium tuberculosis | KasA, KasB | mtFabH |
| Staphylococcus aureus | saFabF | saFabH |
| Streptococcus pneumoniae | spFabF | spFabH |
| Haemophilus influenza | hFabB, hFabF | hFabH |

Thiolactomycin (TLM), specifically inhibits the fatty acid biosynthesis pathway by inhibiting the elongation class of condensing enzymes. TLM was identified in the early 1980s as a novel broad spectrum thiolactone antibiotic that inhibits fatty acid biosynthesis, via inhibition of β-ketoacyl-ACP synthases. It has never been marketed but was reported to effectively protect mice challenged with Serratia marcescens and Klebsiella pneumoniae. Subsequently, TLM has also been shown to possess anti-mycobacterial activity against a variety of M. tuberculosis strains including MDRTB.

Therefore, if appearing herein, the following terms shall have the definitions set out below:

As used herein a “small organic molecule” is an organic compound [or organic compound complexed with an inorganic compound (e.g., metal)] that has a molecular weight of less than 3 Kd and preferably less than 1.5 Kd. Preferably, an agent identified by the methods of the present invention is a small organic molecule.

As used herein, and unless otherwise specified, the terms “agent”, “potential drug”, “test compound” or “potential compound” are used interchangeably, and refer to compounds that potentially have a use as a modulator (and preferably as an inhibitor) of β-ketoacyl-ACP synthase I (FabB). More preferably, an agent is a drug that can be used to treat and/or prevent bacterial infection. Therefore, such “agents”, “potential drugs”, and “potential compounds” may be used, as described herein, in drug assays and drug screens and the like.

Abbreviations used are: ACP, acyl carrier protein; FabB, β-ketoacyl-ACP synthase I; FabF, β-ketoacyl-ACP synthase II; FabH, β-ketoacyl-ACP synthase III; TLM, thiolactomycin, [(4S)(2E,5E)-2,4,6-trimethyl-3-hydroxy-2,5,7-octatriene-4-thiolide]; cerulenin, (2R,3S)-2,3 -epoxy-4-oxo-7,10-dodecandienolyamide.

### Crystallization of the Bacterial -Ketoacyl-[Acyl Carrier Protein] Synthases Complexed with Inhibitors.

The present invention provides β-ketoacyl-ACP synthase inhibitor complexes that have been crystallized into crystals. Preferably such crystals effectively diffract X-rays for the determination of the atomic coordinates of the complex to a resolution of better than 5.0 Angstroms, and more preferably to a resolution equal to or better than 2.3 Angstroms. Of course, the specific β-ketoacyl-ACP carrier protein synthase inhibitor complexes provided herein serve only as examples, since the crystallization process can tolerate a broad range of such complexes. Therefore, any person with skill in the art of protein crystallization having the present teachings, without undue experimentation could crystallize a large number of alternative complexes.

Crystals of the β-ketoacyl-ACP synthase inhibitor complexes of the present invention of the present invention can be grown by a number of techniques including batch crystallization, vapor diffusion (either by sitting drop or hanging drop) and by microdialysis. Seeding of the crystals in some instances may be required to obtain X-ray quality crystals. Standard micro and/or macro seeding of crystals may therefore be used. Exemplified below is the hanging-drop vapor diffusion procedure.

### Protein-structure Based Design of Inhibitors

Once the three-dimensional structure of a crystal comprising a β-ketoacyl-ACP synthase inhibitor complex is determined, (e.g., see the coordinates in Tables III and IV below, in Appendix following the Sequence Listing) other potential modulators of the β-ketoacyl-ACP synthase can be examined through the use of computer modeling using a docking program such as GRAM, DOCK, or AUTODOCK [Dunbrack et al., Folding & Design, 2:27-42 (1997)], to identify potential modulators of the β-ketoacyl-ACP synthase. This procedure can include computer fitting of potential modulators to the β-ketoacyl-ACP synthase to ascertain how well the shape and the chemical structure of the potential modulator will bind to the β-ketoacyl-ACP synthase [Bugg et al., Scientific American, Dec.:92-98 (1993); West et al., TIPS, 16:67-74 (1995)]. Computer programs can also be employed to estimate the attraction, repulsion, and steric hindrance of the complex. In particular, since FabB and FabF are extremely similar, the information provided by the present invention is equally useful for determining inhibitors of either FabB or FabF.

Thus the structural determination disclosed herein allows particular compounds to be selected on the basis of their binding to the active site. Indeed, the disclosure of the atomic coordinates of the individual binding of two different inhibitors to FabB enhances such a determination. Generally the tighter the fit, the lower the steric hindrances, and the greater the attractive forces, the more potent the potential modulator since these properties are consistent with a tighter binding constant. Furthermore, the more specificity in the design of a potential drug the more likely that the drug will not interact as well with other proteins. This will minimize potential side-effects due to unwanted interactions with other proteins.

Initially compounds known to bind β-ketoacyl-ACP synthase, such as thiolactomycin and cerulenin as exemplified below, can be systematically modified by computer modeling programs until one or more promising potential analogs are identified. In addition, systematic modification of selected analogs can then be systematically modified by computer modeling programs until one or more potential analogs are identified. Such analysis has been shown to be effective in the development of HIV protease inhibitors [Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)]. Alternatively a potential modulator could be obtained by initially screening a random peptide library produced by recombinant bacteriophage for example, [Scott and Smith, Science, 249:386-390 (1990); Cwirla et al., Proc. Natl. Acad. Sci., 87:6378-6382 (1990); Devlin et al., Science, 249:404-406 (1990)]. A peptide selected in this manner would then be systematically modified by computer modeling programs as described above, and then treated analogously to a structural analog as described below.

Indeed, high throughput screening for inhibitors of FabH, KasA and KasB can be performed in conjunction with the rational drug design disclosed herein. Potential assay formats include filtration based assays following radiolabel incorporated into trichloroacetic acid insoluble acyl-ACPs, scintillation proximity assays based upon the specific binding of a radiolabled product to a scintillant-containing bead or a coupled FabG (MabA) spectrophotometric assay quantifying NADH/NADPH dependent reduction of the β-ketoacyl-ACP product.

Once a potential modulator/inhibitor is identified it can be either selected from a library of chemicals as are commercially available from most large chemical companies including Merck, GlaxoSmithKline, Bristol Meyers Squib, Monsanto/Searle, Eli Lilly, Novartis and Pharmacia UpJohn, or alternatively the potential modulator may be synthesized de novo. The de novo synthesis of one or even a relatively small group of specific compounds is reasonable in the art of drug design. The potential modulator/inhibitor can be placed into a standard assay as exemplified below.

When suitable potential modulators are identified, a supplemental crystal can be grown which comprises the bacterial β-ketoacyl-ACP synthase and the potential modulator. Preferably the crystal effectively diffracts X-rays for the determination of the atomic coordinates of the protein-ligand complex to a resolution of better than 5.0 Angstroms, more preferably equal to or better than 3.0 Angstroms. The three-dimensional structure of the supplemental crystal can then be determined by Molecular Replacement Analysis. Molecular replacement involves using a known three-dimensional structure as a search model to determine the structure of a closely related molecule or protein-ligand complex in a new crystal form. The measured X-ray diffraction properties of the new crystal are compared with the search model structure to compute the position and orientation of the protein in the new crystal. Computer programs that can be used include: X-PLOR (see above), CNS, (Crystallography and NMR System, a next level of XPLOR), and AMORE [J. Navaza, Acta Crystallographics ASO, 157-163 (1994)]. Once the position and orientation are known an electron density map can be calculated using the search model to provide X-ray phases. Thereafter, the electron density is inspected for structural differences and the search model is modified to conform to the new structure. Using this approach, it will be possible to use the claimed crystal of the bacterial β-ketoacyl-[acyl carrier protein] synthase to solve the three-dimensional structures of other bacterial β-ketoacyl-[acyl carrier protein] synthases having pre-ascertained amino acid sequences. Other computer programs that can be used to solve the structures of the bacterial. β-ketoacyl-[acyl carrier protein] synthases from other organisms include: QUANTA, CHARMM; INSIGHT; SYBYL; MACROMODE; and ICM.

A candidate drug can be selected by performing rational drug design with the three-dimensional structure determined for the supplemental crystal, preferably in conjunction with computer modeling discussed above. The candidate drug (e.g., a potential modulator of a bacterial β-ketoacyl-[acyl carrier protein] synthase) can then be assayed as exemplified above, or in situ. A candidate drug can be identified as a drug, for example, if it inhibits bacterial proliferation.

A potential inhibitor (e.g., a candidate drug) would be expected to interfere with bacterial growth. Therefore, an assay that can measure bacterial growth may be used to identify a candidate drug.

Methods of testing a potential bactericidal agent (e.g., the candidate drug) in an animal model are well known in the art, and can include standard bactericidal assays. The potential modulators can be administered by a variety of ways including topically, orally, subcutaneously, or intraperitoneally depending on the proposed use. Generally, at least two groups of animals are used in the assay, with at least one group being a control group which is administered the administration vehicle without the potential modulator.

For all of the drug screening assays described herein further refinements to the structure of the drug will generally be necessary and can be made by the successive iterations of any and/or all of the steps provided by the particular drug screening assay. More specific drug assays are detailed below.

Assays for FabB/F (Panel A) condensing enzyme: Two FabB and FabF condensation assays can be employed as discussed below. The first is essentially identical to the electrophoretic assay described for FabH in that the substrates, [¹⁴C]malonyl-ACP (50 μM) and myristoyl-ACP (100 μM), are resolved from the product by electrophoresis in a 15% polyacrylamide gel containing 2.5 M urea. The second assay is more appropriate for large numbers of samples, and involves the extraction of the labeled product into toluene for scintillation counting. This assay includes, in a final volume of 20 μl of 0.1 M potassium phosphate, pH 6.8, 100 μM ACP, 1 mM EDTA, 0.3 mM DTT, 1 μg FabD, 50 μM [2-¹⁴C]malonyl-CoA (specific activity 55 mCi/mmole) and 100 μM myristoyl-ACP. The reaction is stopped by the addition of 400 μl of freshly prepared 5 mg/ml sodium borohydride in 30% tetrahydrofuran, 0.4 M potassium chloride to reduce all of the acyl-ACP thioesters to free fatty acid plus ACP-SH. After a 30 min incubation, the mixture is extracted with 400 μl of toluene, and 300 μl of the upper phase counted. This assay can also be used as a scintillation proximity assay. Toluene may be used as the solvent for the scintillation fluor, and a standard toluene-based cocktail to extract the assays and count the mixtures in a scintillation counter to eliminate the sampling step could be used. This assay can be used for screening the TLM analogs, for example and performing the kinetic assays.

Assays for FabH (Panel B) condensing enzymes. Three specific assays of the FabH-catalyzed condensation activity follow. FabH can be most accurately and specifically measured by following the incorporation of radiolabel from acteyl-CoA into acetoacetyl-ACP using a coupled assay containing 25 μM ACP, 1 mM β-mercaptoethanol, 65 μM malonyl-CoA, 45 μM [1-¹⁴C]acetyl-CoA (specific activity 60 mCi/mmole), 3 μg purified FabD, and 1 to 100 ng FabH in a final volume of 40 μL of 0.1 M sodium phosphate, pH 7.0. The ACP is pre-reduced by incubation with the beta-mercaptoethanol for 30 min at 37° C. Reactions are initiated with FabH protein, and incubated at 37° C. for 15 min. The reaction is stopped by pipetting 35 μl onto a Whatmann 3MM filter paper disc, which is then washed successively with 10%, 5% and 1% ice cold trichloroacetic acid. After drying, the radioactivity on each disc is counted by liquid scintillation counting.\

A second FabH assay utilizes a substrate analog, malonyl-CoA and measures the formation of an acetoacetyl-CoA-Mg²⁺ complex spectrophotometrically at 305 nm. The reaction mixture contains 100 μM acetyl-CoA, 5 mM MgCl₂, 50 μg of FabH, and 0.1 M Tris-Cl, pH 7.0 in a final volume of 300 μl. Increase in absorbance is followed for two minutes at 305 nm. Due to the use of the substrate analog, much higher concentrations of protein are required than for the filter disc assay.

A third assay uses electrophoretic separation of the substrate and product. This assay is especially useful when susbtrates other than acetyl-CoA are being tested. An excess of FabG is added to the reaction to convert the unstable β-ketoacyl-ACP product to the more stable β-hydroxyacyl-ACP. Such an assay can contain 50 μM ACP, 100 μM acetyl-CoA (or other primer, such as isovaleryl-CoA), 50 μM [2-¹⁴C]malonyl-CoA (specific activity, 55 mCi/mmole), 3 μg FabD, 3 μg FabG, 1 mM β-mercatoethanol. Reactions are initiated with up to 1 μg of FabH, and incubated at 37° C. for 15 min. The reaction is then placed on ice, gel loading buffer added, and the sample applied to a 13% polyacrylamide gel containing between 0.5 and 1.0 M urea. Following electrophoresis, the gels are dried and exposed to a phosphor storage screen prior to analysis.

The FabH reaction can be split into two half reactions, viz the transfer of the acetyl group to the active site Cys, and the decarboxylation of the malonyl-ACP. Biochemically, the first half reaction can be measured as an exchange of acetyl group from CoA to ACP in an assay identical to the first FabH condensation assay, with the omission of malonyl-CoA and FabD. Decarboxylation assays are performed as for the gel-based FabH assay, but without acetyl-CoA, and using radiolabeled malonyl-CoA.

Antibacterial evaluation: (1) In vitro. Drugs identified can be evaluated in four stages. Primary analysis can be in an OD based assay against six pathogens, including the major organisms of interest described above and an efflux-impaired E.coli mutant. Compounds causing >50% inhibition of growth can be evaluated in a full NCCLS type MIC determination. Compounds demonstrating antibacterial activity above a pre-determined threshold are subject to a secondary profile against up to 100 well characterized isolates representing the species of interest. Those passing this stringent panel can finally be tested against a range of recent clinical isolates, including those resistant at varying levels to currently prescribed antibiotics. Susceptibility to efflux in both Gram-positive pathogens of interest can be evaluated using appropriate mutants and well characterised efflux pump inhibitors.

(2) In vivo. Compounds can be evaluated in animals with the infection models extended to include those suitable for the evaluation of anti-tuberculosis activity, for example. Aerosol infection of mice with M. tuberculosis can be performed. Candidates can be evaluated for efficacy by oral gavage administration for the evaluation of efficacy in animal models of tuberculosis infection up to and including the Guinea pig aerosol model of infection. An ex vivo drug efficacy evaluation model for tuberculosis therapies involving sacrifice and dissection of infected lung and spleen tissue from mice can also be employed. The infected tissue can be homogenized and a candidate compounds evaluated for their ability to restrict the growth of in vivo growing M. tuberculosis.

In vitro potency, specificity and selectivity assays: Potential drugs that are identified, e.g., TLM analogs can be tested for potency and specificity against a variety of bacterial condensing enzymes, including from the following pathogens: M. tuberculosis, S. aureus, E. faecium, S. pneumoniae and H. influenzae

All compounds meeting the IC50 threshold can be assayed against human FAS1, which is the appropriate selectivity screen for inhibitors of fatty acid biosynthesis. Lead compounds can also be evaluated in a cytotoxicity assay using human fibroblasts (A549 cells). Cell viability is determined by measuring the ability of cells to metabolize XTT (a tetrazolium salt). Metabolic degradation of XTT yields a colored, water insoluble formazan salt which can be measured spectrophotometrically. The P450 inhibition potential of lead compounds can be monitored by evaluation against a panel of purified P450 isoforms.

Compounds which have been shown to pass in vitro and in vivo efficacy criteria can be subject to appropriate DMPK evaluation, including half life, mean residence time, clearance, volume of distribution and oral bioavailability.

### Three-Dimensional Representation of the Structure of the FabB-Inhibitor Binding Complex

In addition, the present invention provides a computer that comprises a representation of a FabB-inhibitor binding complex (e.g., a FabB-cerulenin binding complex or FabB-TLM binding complex) in computer memory that can be used to screen for compounds that will or are likely to inhibit FabB. In a related embodiment, the computer can be used in the design of altered FabB's that have either enhanced, or alternatively, diminished synthase activity. Preferably, the computer comprises portions of and/or all of the information contained in Table III and/or Table IV. In a particular embodiment, the computer comprises: (i) a machine-readable data storage material encoded with machine-readable data, (ii) a working memory for storing instructions for processing the machine readable data, (iii) a central processing unit coupled to the working memory and the machine-readable data storage material for processing the machine-readable data into a three-dimensional representation, and (iv) a display coupled to the central processing unit for displaying the three-dimensional representation.

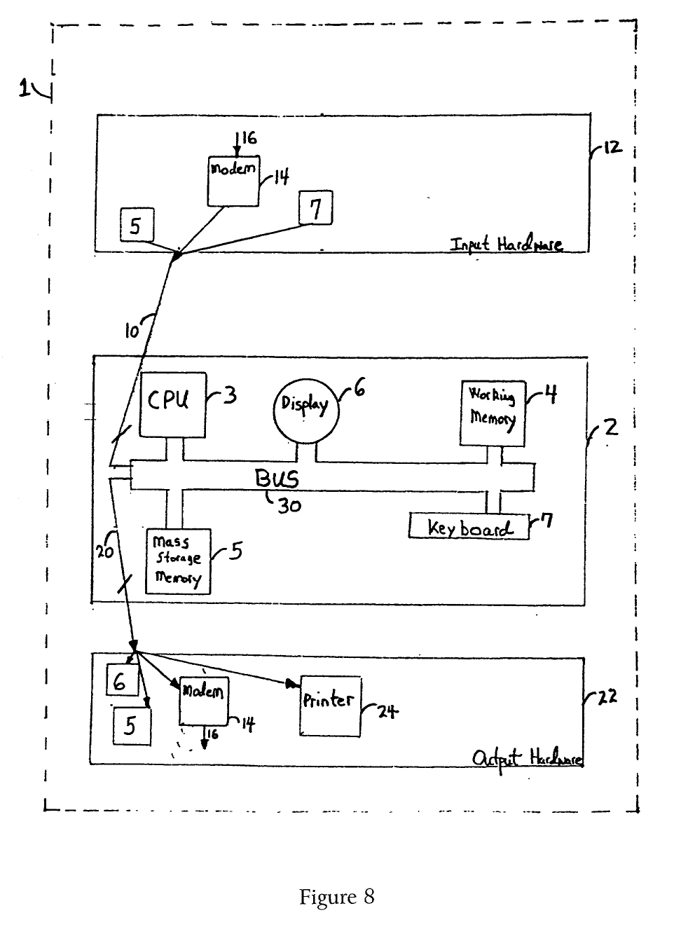

Thus a machine-readable data storage medium comprises a data storage material encoded with machine readable data which can comprise portions and/or all of the structural information contained in Table III and/or Table IV. One embodiment for manipulating and displaying the structural data provided by the present invention is schematically depicted in FIG. 8. As depicted, the System 1, includes a computer 2 comprising a central processing unit (“CPU”) 3, a working memory 4 which may be random-access memory or “core” memory, mass storage memory 5 (e.g., one or more disk or CD-ROM drives), a display terminal 6 (e.g., a cathode-ray tube), one or more keyboards 7, one or more input lines 10, and one or more output lines 20, all of which are interconnected by a conventional bidirectional system bus 30.

Input hardware 12, coupled to the computer 2 by input lines 10, may be implemented in a variety of ways. Machine-readable data may be inputted via the use of one or more modems 14 connected by a telephone line or dedicated data line 16. Alternatively or additionally, the input hardware 12 may comprise CD-ROM or disk drives 5. In conjunction with the display terminal 6, the keyboard 7 may also be used as an input device. Output hardware 22, coupled to computer 2 by output lines 20, may similarly be implemented by conventional devices. Output hardware 22 may include a display terminal 6 for displaying the three dimensional data. Output hardware might also include a printer 24, so that a hard copy output may be produced, or a disk drive 5, to store system output for later use, see also U.S. Pat. No: 5,978,740, Issued Nov. 2, 1999, the contents of which are hereby incorporated by reference in their entireties.

In operation, the CPU 3 (i) coordinates the use of the various input and output devices 12 and 22; (ii) coordinates data accesses from mass storage 5 and accesses to and from working memory 4; and (iii) determines the sequence of data processing steps. Any of a number of programs may be used to process the machine-readable data of this invention.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLE

### INHIBITION OF β-KETOACYL-[ACYL CARRIER PROTEIN] SYNTHASES BY THIOLACTOMYCIN AND CERULENIN:STRUCTURE AND MECHANISM

### EXPERIMENAL PROCEDURES

Materials—Sources of supplies were: Amersham-Pharmcia Biotech., [¹⁴C]malonyl-CoA (specific activity, 55.0 Ci/mole), [¹⁴C]Acetyl-CoA (specific activity, 52.0 Ci/mole); Sigma, ACP, cerulenin; Difco, microbiological media; Promega, molecular reagents; Sigma Chemical Co., cerulenin, and ACP; Qiagen, Ni²⁺-agarose resin; Novagen, pET vector and expression strains; Invitrogen, pCR2.1 vector. Proteins were quantitated by the Bradford method (38) unless otherwise indicated. Acyl-ACP was prepared using an established acyl-ACP synthetase method (14,39,40). The E. coli FabD, FabH, FabB and FabF were purified as described previously (14,41,42). All other supplies were reagent grade or better.

Purification and Assay of Condensing Enzymes—The three condensing enzymes of E. coli were expressed and purified to homogeneity as described previously (13,14,41). Purified enzymes were then dialyzed against 20 mM Tris-HCl, pH 7.6, 1 mM β-mercaptoethanol, 1 mM DTT, concentrated with an Amicon stirred cell, and stored in 50% glycerol at −20° C.

A filter disc assay was used to assay FabH activity with [1-¹⁴C]acetyl-CoA as described previously (15,18). The assays contained 100 μM ACP, 1 mM β-mercaptoethanol, 45 μM [¹⁴C]acetyl-CoA (specific activity, 52.0 Ci/mole), and 50 μM malonyl-CoA, E. coli FabD (0.3 μg) and 0.1 M sodium phosphate buffer (pH 7.0) in a final volume of 40 μl. The reaction was initiated by the addition of FabH and the mixture was incubated at 37° C. for 12 min. A 35-μl aliquot was removed and deposited on a Whatman 3MM filter disc. The discs were washed with three changes (20 ml/disc for 20 min) of ice-cold trichloroacetic acid. The concentration of the trichloroacetic acid was reduced from 10 to 5 to 1% in each successive wash. The filters were dried and counted in 3 ml of scintillation cocktail.

FabB and FabF radiochemical assay was performed using the scheme devised by Garwin et al. (11) using myristoyl-ACP as the substrate. The assays contained 100 μM ACP, 0.3 mM dithiothreitol, 1 mM EDTA, 0.1 M potassium phosphate buffer, pH 6.8, 50 μM [¹⁴C]malonyl-CoA (specific activity 55 Ci/mole), 100 μM myristoyl-ACP, FabD (0.3 μg of protein), in a final volume of 20 μl. A mixture of ACP, 0.3 mM DTT, 1 mM EDTA, and the buffer was incubated at 37° C. for 30 min to ensure complete reduction ACP, and then the remaining components (except the condensing enzyme) were added. The mixture was then aliquoted into the assay tubes and the reaction was initiated by the addition of FabB or FabF. The reaction mixture was incubated at 37° C. for 20 min, then 400 μl of reducing agent (0.1 M K₂HPO₄, 0.4 M KCl, 30% tetrahydrofuran, and 5 mg/ml sodium borohydride) was added into the reaction tubes and incubated for 40 min.

Finally, 400 μl of toluene was added, vigorously mixed, and 300 μl of upper phase solution was counted in 3 ml of scintillation cocktail.

Construction of the FabB[H333N] mutant—A portion of the fabB gene was amplified using the polymerase chain reaction with two specific primers. The first primer introduced the desired mutation, and extended over a unique AgeI site (5′-AAAGCCATGACCGGTAACTCTC-3′, SEQ ID NO:3). The second primer created a BamHI site downstream of the stop codon (5′-GCAGGATCCGGCGATTGTCAATGATG-3′, SEQ ID NO:4). The resulting fragment was sequenced to confirm that the mutation had been correctly introduced, and then digested with AgeI and BamHI and cloned into the pET-15b-FabB expression vector that had been digested with the same enzymes. The FabB[H333N] protein was expressed and purified as described above.

Structure Determination of the FabB-Cerulenin and FabB-TLM Complexes—Both structures were solved using electron density difference maps. Pure FabB protein was dialyzed (10 mM Tris-HCl, pH 8.0, 1 mM DTT, 1 mM EDTA) and concentrated to 15 mg/ml. The 403 amino acid residues seen in the structure (Tables III and IV) are included in SEQ ID NO:1. Thus, SEQ ID NO:1 contains residue number 2 to number 404 of the E.coli FabB. [Residue number 1 is a methionine (Met, M), residue 405 is a Lysine (Lys, K) and residue 406 is aspartic acid (Asp, D).]

The inhibitors (TLM and cerulenin) were added directly to separate aliquots of the protein solution and gently agitated for 1 hour. The ratio of inhibitor molecules to FabB monomers was about 10:1.

Crystallization of the FabB-Cerulenin and FabB-TLM Complexes—Pure FabB protein was dialyzed (10 mM Tris-HCl, pH 8.0, 1 mM DTT, 1 mM EDTA) and concentrated to 15 mg/ml. The inhibitors (TLM and cerulenin) were added directly to separate aliquots of the protein solution and gently agitated for 1 hour. The ratio of inhibitor molecules to FabB monomers was about 10:1. Crystals of the FabB:antibiotic complexes were crystallized at 18° C. by the hanging drop vapor diffusion method. The well solution contained 2.0 M ammonium sulfate, 20% PEG 400, and 100 mM Tris pH 6.5. The drops consisted of 5 μl of protein solution and 5 μl of well solution. Crystals measuring 0.1 mm×0.3 mm×1.0 mm grew in one to two weeks. The crystals were mounted on standard nylon loops, passed through a cryoprotectant of 50% paratone-N, 50% mineral oil, and frozen directly in liquid nitrogen. Data were collected at 100 K using a Nonius FR591 X-ray generator and DIP 2030H detector system. All diffraction data were integrated using the HKL software package (44). Integrated data were merged and scaled using SCALEPACK. Crystals of both complexes have space group P2₁2₁2₁ and cell dimensions similar to the native crystals (Tables I and II).

All refinements of models against the data were carried out using XPLOR (45). First, the native FabB structure (21) was refined against the data, and then 2mFₒ-DF_{c} maps were calculated using CCP4 programs (46). To optimize the maps, the program DM (47) was used to perform histogram matching, solvent flattening and 4-fold NCS averaging (there are 2 dimers in the asymmetric unit). Maps were examined using the program O (48), and determined to be of good quality. Both maps clearly showed the presence of an antibiotic in the active site. The three dimensional structures of the inhibitors were fit by hand into the electron density of one monomer, and then extended by NCS operators into the other sites. For both antibiotics, all four sites showed a good fit between the map and the hand-fit molecule. Waters were picked using XPLOR, and were visually inspected for good electron density and for sensible H-bonding geometry. Incorrectly assigned waters were rejected and some additional waters were added by hand. Full scale refinements using NCS restraints were then performed. The residues to include in NCS restraints were chosen to be those residues not involved in crystal contacts, as determined by the XPLOR script “geomanal,” and by a visual inspection of crystal packing. Two to three cycles of refinement followed by manual rebuilding of each model completed the structure determinations. The statistics of the final models are shown in Tables I and II.

### RESULTS

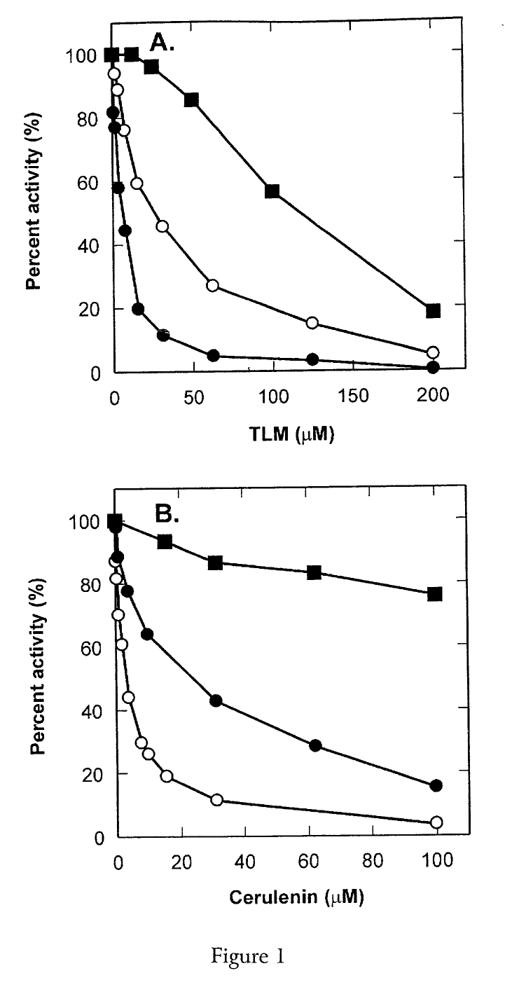

Inhibition of Condensing Enzymes by TLM and Cerulenin—The relative sensitivities of the condensing enzymes to TLM and cerulenin are known from the inhibition of the pathway in crude cell extracts, and from analyses of growth inhibition in genetically modified E. coli strains. However, the activities of the purified enzymes have not been compared using natural substrates. Therefore, the IC₅₀ values were determined for all three condensing enzymes for both TLM and cerulenin (FIG. 1). Each of the three condensing enzymes was inhibited by TLM (FIG. 1A). FabF was the most sensitive enzyme (IC₅₀=6 μM) followed by FabB (IC₅₀=25 μM), and FabH, which was considerably less sensitive (IC₅₀=110 μM). These data are consistent with genetic experiments which show that overexpression of FabB confers TLM resistance whereas FabH overexpression does not (49). Increased expression of FabF blocks growth (50) precluding a similar experiment with this condensing enzyme. However, since FabF is not essential for the growth of E. coli (11), FabB is the physiologically relevant TLM target in this bacterium.

As expected (51), both FabB and FabF were inhibited by cerulenin, with FabB being the most sensitive enzyme (FIG. 1B). This is consistent with previous work that examined fatty acid production in resistant bacteria (which overexpress either FabB or FabF), and which indicated that FabB was the more sensitive of the two enzymes in vivo. FabH was essentially resistant to cerulenin, as reported previously for cell extracts (17).

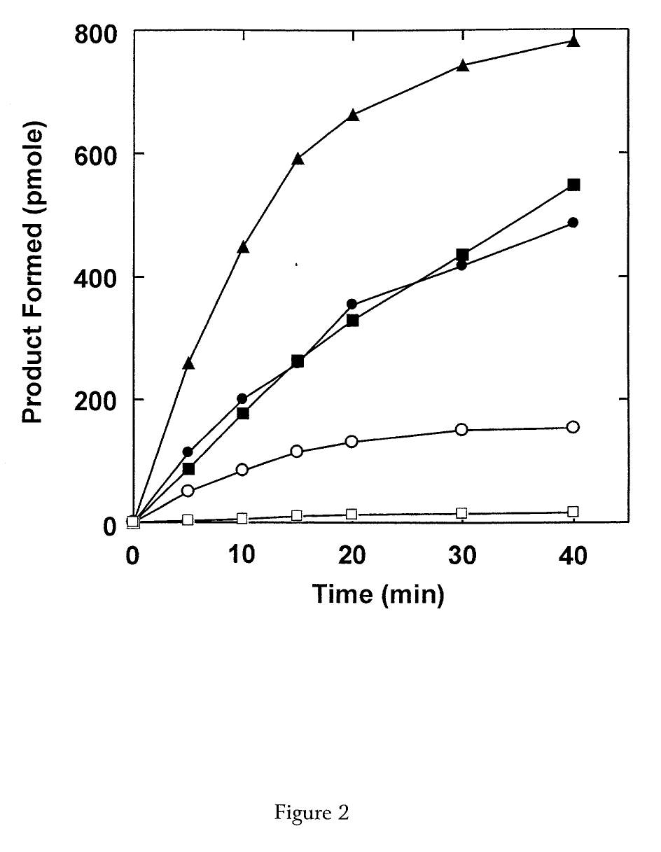

TLM is a Reversible Inhibitor—The thiolactone structure in TLM suggested that it may form a covalent adduct with the condensing enzyme via a thioester exchange reaction. This hypothesis was examined by comparing the kinetics of TLM inhibition to that of cerulenin (FIG. 2), which is known to form a covalent complex with the active site cysteine of the condensing enzymes. The kinetics of cerulenin inhibition exhibited the hallmarks of a slow-binding, irreversible inhibitor (52). When the reaction was initiated with enzyme, there was an initial burst of product formation, but the FabB reaction rate rapidly decreased and ceased by 20 min (FIG. 2B). When cerulenin was preincubated with FabB and the reaction was initiated by the addition of the substrates, there was no discernable product formation. This indicates the formation of the irreversible FabB-cerulenin binary complex. The same two types of time course experiments were performed with TLM (FIG. 2A). There was no evidence for the formation of an irreversible or slow-binding FabB-TLM complex, and it therefore may be concluded that TLM is a reversible inhibitor of FabB.

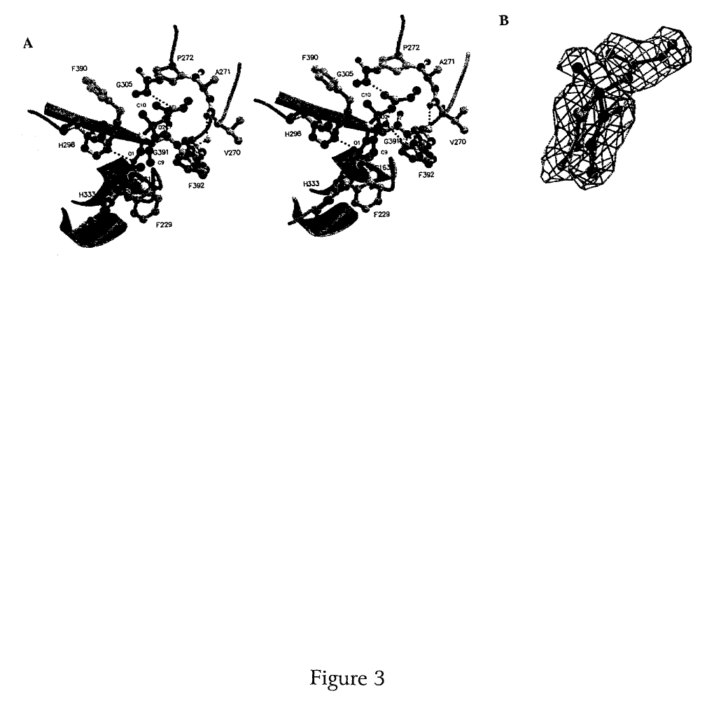

The Structure of the FabB-TLM Binary Complex—TLM makes a number of specific but non-covalent interactions within the FabB active site (FIG. 3). The C9 and C10 methyl groups are nestled within two hydrophobic pockets comprising phenylalanines 229 and 392, and Pro272 and Phe390, respectively. The isoprenoid moiety is wedged between two peptide bonds, 391-392 ‘below’ and 271-272 ‘above’, and this intercalated stacking interaction between three delocalized systems is clearly an important element of specificity. The ring of Pro272 participates in this molecular sandwich by van der Waals interactions. The O1 and O2 exocyclic oxygens are both involved in hydrogen bonding interactions. O1 interacts with the Nε2 nitrogens of the two active site histidines 298 and 333, and O2 bonds to the carbonyl oxygen of residue 270 and the amide nitrogen of residue 305 through a lattice of water molecules. These water molecules are at the base of the active site tunnel, and the O2 oxygen is appropriately oriented towards the tunnel. Finally, the thiolactone sulfur does not make any obvious specific interactions, but it is adjacent to the active site cysteine 163.

The exquisite fit of TLM into the FabB active site is reflected in the minimal distortion it causes to the native FabB structure. The movements that do occur involve local changes in the FabB main chain positions, but these do not extend beyond the immediate vicinity of the active site (FIG. 3). The sidechain of Cys163 shifts by 2.1 Å to avoid a clash with the TLM sulfur, and His298 moves by 2 Å to improve the hydrogen bonding geometry to the O1 of TLM. Finally, phenylalanines 390 and 392, and the associated loop comprising residues 388-394, all move by about 1.0 Å, and the side chain of Phe392 also rotates ˜40°. These latter movements correlate with the new location of His298. A hydrogen bonding interaction between the Nε2 nitrogen and the carbonyl oxygen of residue 390 fixes the orientation of the histidine. When the histidine moves, the loop also moves to maintain this important interaction.

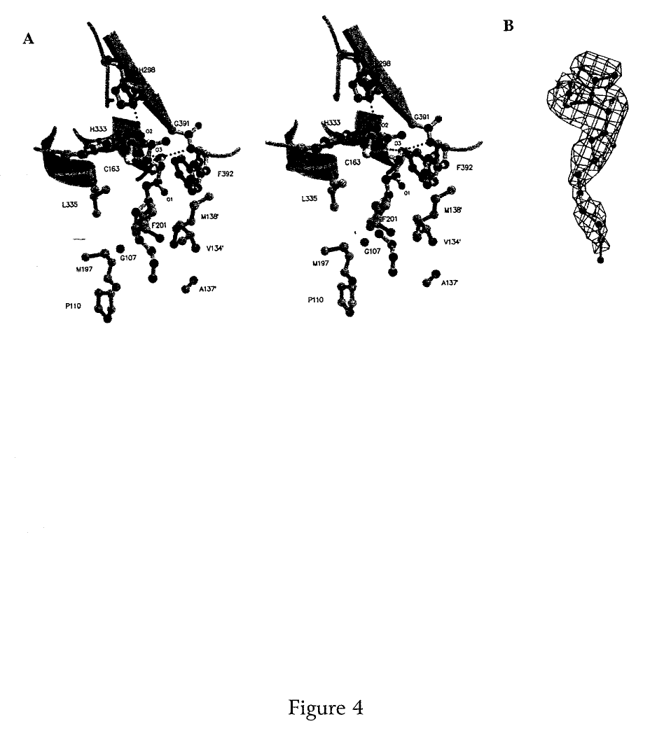

The Structure of the FabB-Cerulenin Complex—In contrast to TLM, cerulenin forms a covalent complex with FabB (FIG. 4). Also, with one major exception, it occupies a completely different region of the active site. The covalent bond is formed between the central C2 carbon of cerulenin and the active site Cys163. The O2 and O3 oxygens are crucial specificity determinants that form important hydrogen bonds. O2 interacts with the Nε2 nitrogens of the active site histidines 298 and 333, and is in the same position as the O1 of TLM. O3 is hydrogen bonded to the amide nitrogens of residues 163 and 392. The O1 oxygen and N1 nitrogen do not form hydrogen bonds, but likely interact with the π electrons of the adjacent Phe292 side chain. Finally, the extended acyl chain of cerulenin is located within a deep hydrophobic pocket at the dimer interface comprising Gly107, Pro110, Val134′ (prime refers to the other monomer), alanines 137′ and 162, methionines 138′ and 197, Phe201 and Leu335. No electron density was observed for C12, indicating that the end of the cerulenin chain may be flexible.

Like TLM, cerulenin occupies the active site with minimal distortion of the surrounding FabB structure, and the slight movements that do occur are similar to those observed in the TLM complex. Thus, Cy163 moves to form the covalent bond, histidines 298 and 333 move to optimize the hydrogen bond interactions with the O2, and phenylalanines 390 and 392 and their associated loop shift by ˜1.5 Å in concert with the movement of His298. Notably, residues in the deep hydrophobic pocket do not move, suggesting that the acyl chain has evolved to optimize this fit.

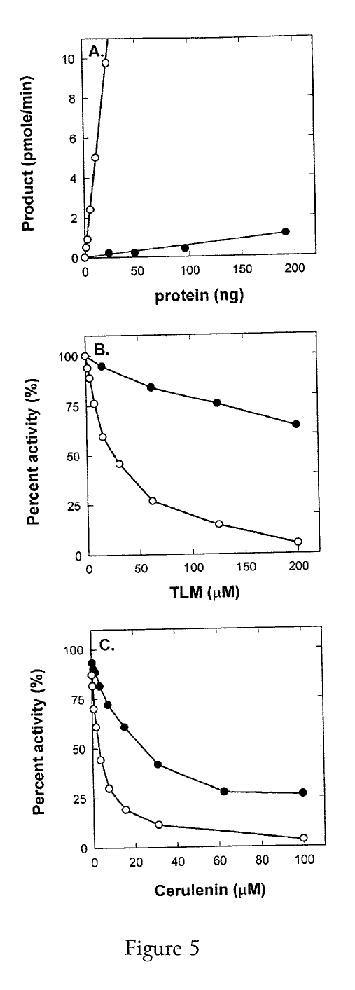

Importance of His-His Active Site Configuration in TLM Inhibition—The structural information indicates that the strong hydrogen bond interactions between the two active site histidines and the O1 of TLM are important determinants of high affinity TLM binding. The FabH condensing enzyme has a His-Asn configuration and was much less sensitive to TLM (FIG. 1). The importance of the two histidines was directly addressed by creating the FabB[H333N] mutant which converts the FabB active site into a FabH configuration. The specific activity of FabB [H333N] was reduced compared to FabB, but it still retained a significant condensation activity (FIG. 5A). However, FabB[H333N] was significantly more resistant to inhibition by both TLM (FIG. 5B) and cerulenin (FIG. 5C). These data indicate that the two-histidine active site architecture is an important determinant of the reactivity of condensing enzymes toward both TLM and cerulenin, and that it contributes to the observed resistance of the FabH class of enzymes to these two antibiotics (FIG. 1).

### DISCUSSION

The structural and biochemical analyses of the binding of TLM and cerulenin to FabB provide the framework for understanding the specificity of these antibiotics and provide clues for the development of more potent compounds that target type II fatty acid synthesis. Although the condensation enzymes have similar active sites, subtle structural variations define their differential response to these molecules. The sensitivity of the condensing enzymes to TLM inhibition is FabF>FabB>>FabH. The interactions that account for the slight difference in TLM binding to FabF and FabB is not clear. In contrast, there are two clear reasons why TLM should be a poor inhibitor of FabH. The first is that histidines 298 and 333 in FabB are replaced by His244 and Asn274 in FabH, and our FabB-TLM structure reveals that the two histidines form strong hydrogen bonds with the antibiotic (FIG. 3). To test the importance of this interaction, the FabB active site was converted into a FabH active site by constructing the FabB[H333N] mutant, showing that the absence of the histidine dyad imparts resistance to TLM (FIG. 5). The structural basis of this resistance is not entirely clear since the asparagine is capable of promoting the condensation reaction (in FabH), and could presumably donate a hydrogen bond to O1 of TLM. The second reason why TLM is a poor inhibitor of FabH is the absence of the peptide bond ‘sandwich’ that binds the isoprenoid group. Specifically, there is no equivalent in FabH of the loop containing Pro272, and the sandwich cannot form.

The isoprenoid moeity in TLM takes advantage of a specific hydrophobic crevice that is present in the active sites of both FabB and FabF. However, these hydrophobic pockets extend further back into the proteins' interiors, and are not optimally filled by the TLM side chain. This would explain the results of inhibition studies against plant (53) and mycobacterial (54) fatty acid synthase systems that used TLM analogs in which the isoprenoid was replaced by various acyl chains. Analogs with longer, more flexible chains showed increased activity against FAS II in both organisms, and these longer chains may more completely fill the available space. Also, TLM analogs with shorter chains lacking the double bond are less active. It is worth noting that the analog studies assumed that the isoprenoid chain binds in the natural substrate pocket, and the structural studies disclose herein clearly show that this is not the case. This structural insight has important implications for the design of more potent inhibitors against these enzymes.

In the case of cerulenin, the order of inhibition is FabB>FabF>>FabH. One reason cerulenin is a poor inhibitor of FabH is the fact that FabH lacks the substrate hydrophobic pocket to accommodate the acyl chain of the drug. This pocket is not required in E. coli FabH where the substrate acyl group is simply the initiating acetyl moiety. However, long-chain acyl groups are accommodated by M. tuberculosis FabH (16), and this enzyme is still resistant to cerulenin (16). This supports the finding that the His-His active site, as opposed to the FabH His-Asn active site configuration, is crucial for optimal cerulenin inhibition (FIG. 5). The reason why FabB is more susceptible than FabF is understood by comparing the FabB-cerulenin complex (FIG. 4) with the FabF-cerulenin complex (23). In the FabB-cerulenin complex, Gly107 and Met197 face each other in the substrate hydrophobic pocket and direct the cerulenin acyl chain towards strand β4 (to the back in FIG. 4). However, in the FabF-cerulenin complex, the steric configuration is reversed, and Ile108 and Gly198 direct the cerulenin tail away from strand β34 (to the front in FIG. 4). Furthermore, Ile108 must swing around to accomodate the acyl chain in FabF, but Met197 does not move in FabB. This required movement in FabF probably explains why cerulenin is a better inhibitor of FabB than of FabF. It is also possible that this structural difference between FabF and FabB relates to the differences in their substrate specificities and physiological functions. When the cerulenin structures of FabB and FabF are superimposed, they are identical except for the acyl chain which adopts these different positions. FabB catalyzes reactions in the elongation of short-chain unsaturated fatty acid intermediates, and its active site must accommodate acyl chains with the characteristic kink imposed by the cis double bond. In contrast, FabF does not accept these intermediates. Thus, these cerulenin bound structures provide a framework for modifying the substrate binding pocket by site directed mutagenesis to define the important differences in the elongation condensing enzymes that define their physiological functions.

It is clear that FabB is the physiologically important TLM target in E. coli since FabF is not an essential enzyme (11), and elevated expression of the fabB gene confers TLM resistance whereas increased levels of FabH does not (49). In contrast, FabF-like proteins are the only elongation condensing enzymes expressed in many pathogens, such as Streptococcus pneumoniae and Staphylococcus aureus, and it is likely that FabF is the relevant TLM target in these organisms. Bacillus subtilis stands out as an organism that is uniquely resistant to TLM (55,56). Accordingly, TLM is a very weak inhibitor of B. subtilis FabF in vitro.

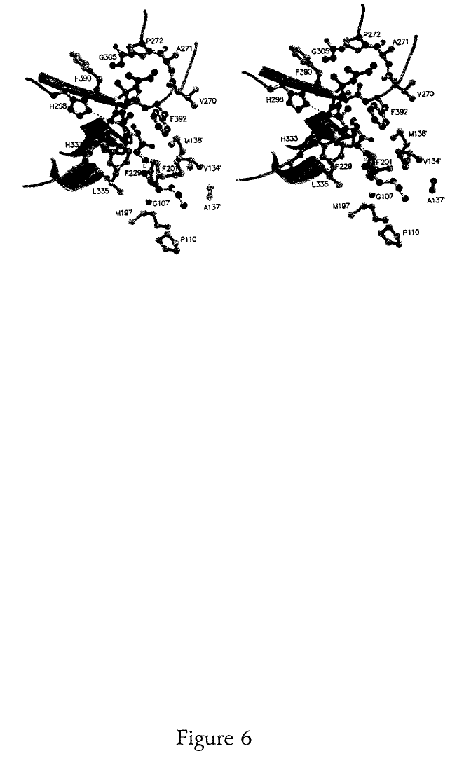

The structures of the FabB-TLM and FabB-cerulenin complexes support the division of the enzymes' common active site into a transacylation half and a decarboxylation half based on our site-directed mutagenesis work (20). The location of the two antibiotics with respect to the two sides of the FabB active site is clearly seen in an overlay of the FabB-TLM and FabB-cerulenin structures (FIG. 6). The tail of cerulenin occupies the hydrophobic cavity which accommodates the fatty acid chain of the acyl-enzyme intermediate. On the other hand, the ring of TLM takes the place of the incoming malonyl group which participates in the decarboxylation reaction. The O1 of TLM and O2 of cerulenin are the only two atoms that occupy the same space in the two structures. In both cases, these carbonyl oxygens form strong hydrogen bonds with the active site histidine dyad, an important component of antibiotic interactions with the enzyme (FIG. 6). It is interesting to note that the movements in the FabB active site caused by TLM and cerulenin are very similar. The fact that these movements directly relate to the inhibitors' hydrogen bonding interactions with the histidine dyad, suggests that they also occur during substrate binding, and are part of the active site mechanism. One possibility is that they represent communication between the two half sites that promotes the ping pong mechanism.

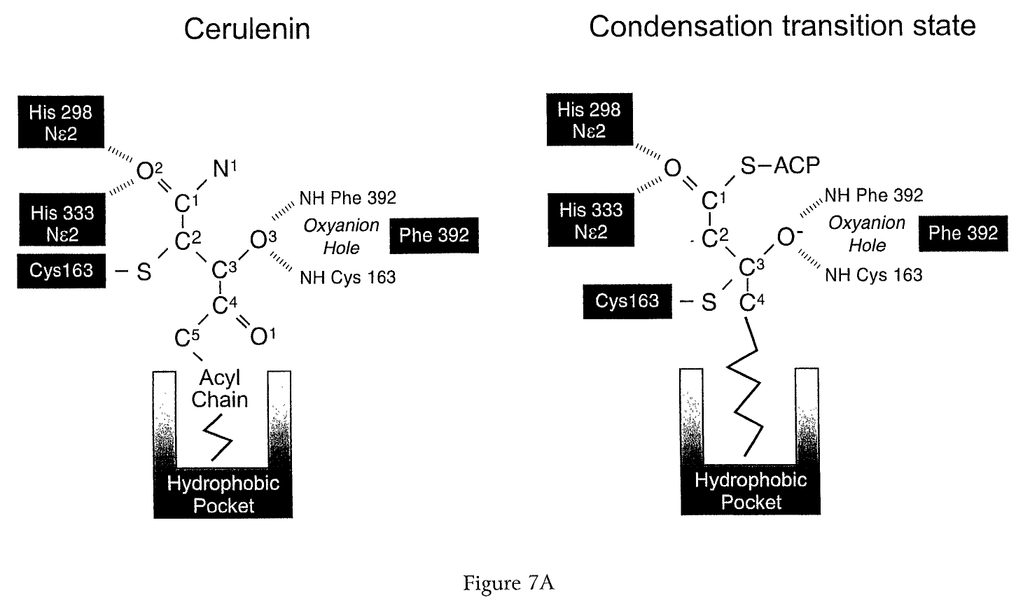

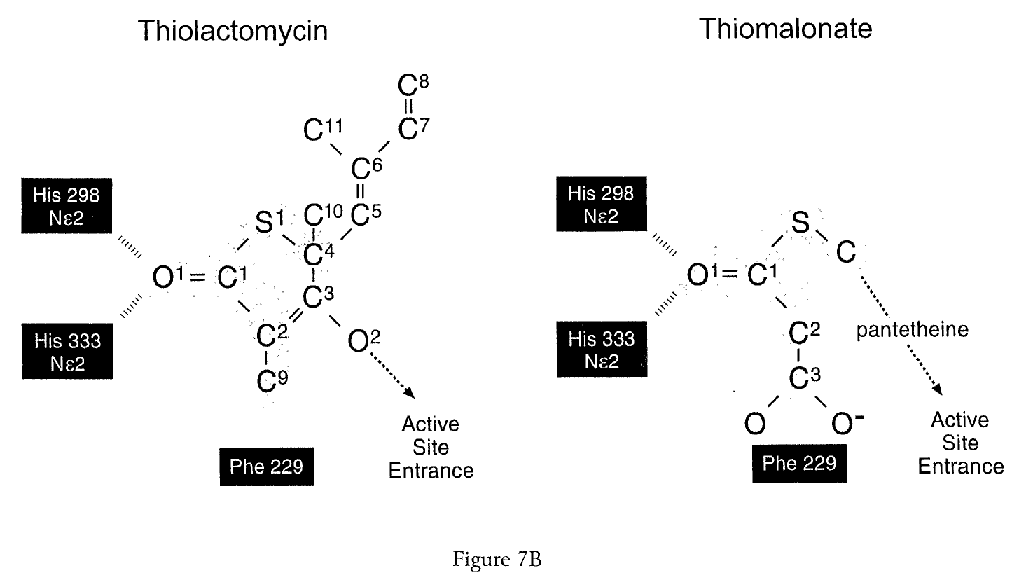

Cerulenin mimics the condensation transition state (FIG. 7A). The C2 carbon forms a covalent bond with the active site cysteine, the O3 oxygen mimics the substrate oxygen in the oxyanion hole formed by the amide nitrogens of residues 163 and 392, and the O2 oxygen represents the carbonyl oxygen of the incoming malonyl group. The acyl chain mimics the location of the acyl-enzyme intermediate, and its location identifies the hydrophobic substrate-binding pocket.

In contrast, TLM mimics the non-covalently bound thiomalonate that enters the active site after the formation of the acyl-enzyme intermediate to participate in the decarboxylation/condensation half step of the reaction (FIG. 7B). During the decarboxylation reaction, it was proposed (20) that the thiomalonate is oriented such that the thioester carbonyl oxygen is hydrogen bonded to the two acceptors (histidines 298 and 333 in the case of FabB), and the terminal carboxyl group is adjacent to a conserved phenylalanine (Phe229 in the case of FabB). This promotes the movement of electrons away from the carboxyl group during decarboxylation and the formation of a carbanion at C2 of malonate. This scheme requires that the thiomalonate be bent within the active site, and the TLM thiolactone ring mimics this bent structure. Thus, the ring sulfur, the O1 oxygen and the C9 carbon of TLM appear to corresponds to the positions of the thiol, the carbonyl oxygen, and the carboxylate carbon of the malonyl-ACP substrate, respectively. The isoprenoid TLM side-chain nestles into a hydrophobic side pocket in the active site tunnel and does not represent a structural feature of the malonyl-ACP substrate.

### REFERENCES

1. Smith, S. (1994) FASEB J. 8, 1248-1259

2. Rock, C. O. and Cronan, J. E., Jr. (1996) Biochim. Biophys. Acta 1302, 1-16

3. Cronan, J. E., Jr. and Rock, C. O. (1996) Biosynthesis of membrane lipids. In Neidhardt, F. C., Curtis, R., Gross, C. A., Ingraham, J. L., Lin, E. C. C., Low, K. B., Magasanik, B., Reznikoff, W., Riley, M., Schaechter, M., and Umbarger, H. E., editors. Escherichia coli and Salmonella typhimurium: cellular and molecular biology, American Society for Microbiology, Washington, D.C.

4. McMurray, L. M., Oethinger, M., and Levy, S. (1998) Nature (London) 394, 531-532

5. Heath, R. J., Yu, Y.-T., Shapiro, M. A., Olson, E., and Rock, C. O. (1998) J. Biol. Chem. 273, 30316-30321

6. Dessen, A., Quémard, A., Blanchard, J. S., Jacobs, W. R., Jr., and Sacchettini, J. C. (1995) Science 267, 1638-1641

7. Rosenfeld, I. S., D'Agnolo, G., and Vagelos, P. R. (1973) J. Biol. Chem. 248, 2452-2460

8. D'Agnolo, G., Rosenfeld, I. S., and Vagelos, P. R. (1975) J. Biol. Chem. 250, 5289-5294

9. Garwin, J. L., Klages, A. L., and Cronan, J. E., Jr. (1980) J. Biol. Chem. 255, 3263-3265

10. de Mendoza, D. and Cronan, J. E., Jr. (1983) TIBS 8,49-52

11. Garwin, J. L., Klages, A. L., and Cronan, J. E., Jr. (1980) J. Biol. Chem. 255, 11949-11956

12. Gelmann, E. P. and Cronan, J. E., Jr. (1972) J. Bacteriol. 112, 381-387

13. Heath, R. J. and Rock, C. O. (1996) J. Biol. Chem. 271, 1833-1836

14. Heath, R. J. and Rock, C. O. (1996) J. Biol. Chem. 271, 10996-11000

15. Choi, K.-H., Heath, R. J., and Rock, C. O. (2000) J. Bacteriol. 182, 365-370

16. Choi, K.-H., Kremer, L., Besra, G. S., and Rock, C. O. (2000) J. Biol. Chem. 275, (in press)

17. Jackowski, S. and Rock, C. O. (1987) J. Biol. Chem. 262, 7927-7931

18. Tsay, J.-T., Oh, W., Larson, T. J., Jackowski, S., and Rock, C. O. (1992) J. Biol. Chem. 267, 6807-6814

19. Qiu, X., Janson, C. A., Konstantinidis, A. K., Nwagwu, S., Silverman, C., Smith, W. W., Khandekar, S., Lonsdale, J., and Abdel-Meguid, S. S. (1999) J. Biol. Chem. 274, 36465-36471

20. Davies, C., Heath, R. J., White, S. W., and Rock, C. O. (2000) Structure. 8, 185-195

21. Olsen, J. G., Kadziola, A., Wettstein-Knowles, P., Siggaard-Andersen, M., Lindquist, Y., and Larsen, S. (1999) FEBS Lett. 460, 46-52

22. Huang, W., Jia, J., Edwards, P., Dehesh, K., Schneider, G., and Lindqvist, Y. (1998) EMBO J. 17, 1183-1191

23. Vance, D. E., Goldberg, I., Mitsuhashi, O., Bloch, K., Omura, S., and Nomura, S. (1972) Biochem. Biophys. Res. Commun. 48, 649-656

24. D'Agnolo, G., Rosenfeld, I. S., Awaya, J., Omura, S., and Vagelos, P. R. (1973) Biochim. Biophys. Acta 326, 155-166

25. Kawaguchi, A., Tomoda, H., Nozoe, S., Omura, S., and Okuda, S. (1982) J. Biochem. (Tokyo) 92, 7-12

26. Kauppinen, S., Siggaard-Anderson, M., and van Wettstein-Knowles, P. (1988) Carlsberg. Res. Commun. 53, 357-370

27. Omura, S. (1976) Microbiol. Rev. 40, 681-697

28. Omura, S. (1981) Methods Enzymol. 72, 520-532

29. Kuhajda, F. P., Pizer, E. S., Li, J. N., Mani, N. S., Frehywot, G. L., and Townsend, C. A. (2000) Proc. Natl. Acad. Sci. U. S. A 97, 3450-3454

30. Loftus, T. M., Jaworsky, D. E., Frehywot, G. L., Townsend, C. A., Ronnett, G. V., Lane, M. D., and Kuhajda, F. P. (2000) Science 288, 2379-2381

31. Hayashi, T., Yamamoto, O., Sasaki, H., and Okazaki, H. (1984) J. Antibiot. (Tokyo) 37, 1456-1461

32. Hayashi, T., Yamamoto, O., Sasaki, H., Kawaguchi, A., and Okazaki, H. (1983) Biochem. Biophys. Res. Commun. 115, 1108-1113

33. Miyakawa, S., Suzuki, K., Noto, T., Harada, Y., and Okazaki, H. (1982) J. Antibiot. (Tokyo) 35, 411-419

34. Hamada, S., Fujiwara, T., Shimauchi, H., Ogawa T, Nishihara, T., Koga, T., Neheshi, T., and Matsuno, T. (1990) Oral Microbiol. Immunol. 5, 340-345

35. Slayden, R. A., Lee, R. E., Armour, J. W., Cooper, A. M., Orme, I. M., Brennan, P. J., and Besra, G. S. (1996) Antimicrob. Agents Chemother. 40, 2813-2819

36. Wailer, R. F., Keeling, P. J., Donald, R. G. K., Striepen, B., Handman, E., Kang-Unnasch, N., Cowman, A. F., Besra, G. S., Roos, D., and McFadden, G. I. (1998) Proc. Natl. Acad. Sci. U. S. A. 95, 12352-12357

37. Morita, Y. S., Paul, K. S., and Englund, P. T. (2000) Science 288, 140-143

38. Bradford, M. M. (1976) Anal. Biochem. 72, 248-254

39. Rock, C. O., Garwin, J. L., and Cronan, J.E., Jr. (1981) Methods Enzymol. 72, 397-403

40. Rock, C. O. and Garwin, J. L. (1979) J. Biol. Chem. 254,7123-7128

41. Heath, R. J. and Rock, C. O. (1995) J. Biol. Chem. 270, 26538-26542

42. Heath, R. J. and Rock, C. O. (1996) J. Biol. Chem. 271, 27795-27801

43. Zhang, Y. M., Rao, M. S., Heath, R. J., Price, A. C., Olson, A. J., Rock, C. O., and White, S, (2001) J. Biol. Chem. 276, 8231-8238

44. Otwinowski, Z. (1993) Data collection and processing. In Sawyer, L., Isaacs, N., and Bailey, S., editors. Proceedings of the CCP4 study weekend, SERC Daresbury Laboratory, Warrington, UK

45. Brünger, A. T. (1992) X-PLOR, a system for X-ray crystallography and NMR, Yale University Press, New Haven, Conn.

46. Collaborative Computation Project, N. 4. (1994) Acta Crystallogr. D 50, 760-763

47. Cowtan, K. (1994) Joint CCP4 and ESF-EACBM Newsletter on Protein Crystallography 31, 34-38

48. Jones, T. A., Zou, J. Y., Cowan, S. W., and Kjeldgaard, M. (1991) Acta Crystallogr. A 47,110-119

49. Tsay, J.-T., Rock, C. O., and Jackowski, S. (1992) J. Bacteriol. 174, 508-513

50. Subrahmanyam, S. and Cronan, J. E., Jr. (1998) J. Biol. Chem. 180, 4596-4602

51. Zhou, Y. and Bowie, J. U. (2000) J. Biol. Chem. 275,6975-6979

52. Morrison, J. F. and Walsh, C. T. (1988) Adv. Enzymol. 61, 201-301

53. Jones, A. L., Herbert, D., Rutter, A. J., Dancer, J. E., and Harwood, J. L. (2000) Biochem. J. 347, 205-209

54. Kremer, L., Douglas, J. D., Baulard, A. R., Morehouse, C., Guy, M. R., Alland, D., Dover, L. G., Lakey, J. H., Jacobs, W. R., Jr., Brennan, P. J., Minnikin, D. E., and Besra, G. S. (2000) J. Biol. Chem. 275, 16857-16864

55. Noto, T., Miyakawa, S., Oishi, H., Endo, H., and Okazaki, H. (1982) J. Antibiot. (Tokyo) 35, 401-410

56. Arimyra, N. and Kaneda, T. (1993) Arch. Microbiol. 160, 158-161

TABLE I
Statistics of data collection and refinement for
the FabB-TLM binary complex

| | Parameter | |
|---|---|---|
| Data Collection | | |
| Space group | P2₁2₁2₁ | |
| Cell dimensions (Å) | a = 59.1, b = 139.0, | |
| | c = 211.9 | |
| Resolution range (Å) | 19.96-2.35 | (240-235)^{b} |
| Multiplicity | 4.1 | (3.7) |
| R_{sym}^{a} | 14.8 | (56.6) |
| I/□ | 8.3 | (2.0) |
| Completeness (%) | 95.5 | (92.1) |
| Reflections | 626204 | |
| Unique reflections | 75201 | |
| Refinement | | |
| Number of reflections in working set (R_{work}) | 69481 | |
| Number of reflections in test set (R_{free}) | 5720 | |
| Number of protein atoms in asymmetric unit | 11824 | |
| Number of TLM atoms in asymmetric unit | 56 | |
| Number of water molecules in | 306 | |
| asymmetric unit | | |
| R_{work} (%) | 19.7 | |
| R_{free} (%) | 25.3 | |
| rms deviations from ideal stereochemistry:- | | |
| Bond lengths (Å) | 0.007 | |
| Bond angles (°) | 1.35 | |
| Dihedrals (°) | 26.3 | |
| Impropers (°) | 0.70 | |
| Mean B factor (main chain) (Å²) | 19.2 | |
| rms deviation in main chain B factor (Å²) | 5.3 | |
| Mean B factor (side chains and waters) (Å²) | 19.3 | |
| rms deviation in side chain B factors (Å²) | 7.9 | |
| Ramachandran plot: | | |
| Residues in most favored region (%) | 89.1 | |
| Residues in additionally allowed region (%) | 9.4 | |
| Residues in generously allowed regions (%) | 1.2 | |
| Residues in disallowed regions (%) | 0.3 | (1 res.) |

^{a}R_{SYM} = ΣΣ|Iᵢ − Iₘ|/ΣΣ Iᵢ where Iᵢ is the intensity of the measured reflection and Iₘ is the mean intensity of all symmetry-related reflections.
^{b}Parameters in parenthesis refer to the outer resolution shell.

TABLE II
Statistics for data collection and refinement
of the FabB-cerulenin binary complex.

| | Parameters | |
|---|---|---|
| Data Collection | | |
| Space group | P2₁2₁2₁ | |
| Cell dimensions (Å) | a = 59.2, b = 139.6, | |
| | c = 212.2 | |
| Resolution range (Å) | 19.88-2.27 | (2.31-2.27)^{b} |
| Multiplicity | 4.1 | (3.6) |
| R_{sym}^{a} | 9.2 | (33.9) |
| I/□ | 17.0 | (3.7) |
| Completeness (%) | 97.3 | (91.8) |
| Reflections | 741083 | |
| Unique reflections | 79821 | |
| Refinement | | |
| Number of reflections in working set (R_{work}) | 71817 | |
| Number of reflections in test set (R_{free}) | 8004 | |
| Number of protein atoms in asymmetric unit | 11824 | |
| Number of CER atoms in asymmetric unit | 64 | |
| Number of water molecules in | 295 | |
| asymmetric unit | | |
| R_{work} (%) | 23.0 | |
| R_{free} (%) | 26.5 | |
| rms deviations from ideal stereochemistry:- | | |
| Bond lengths (Å) | 0.008 | |
| Bond angles (°) | 1.39 | |
| Dihedrals (°) | 26.3 | |
| Impropers (°) | 0.74 | |
| Mean B factor (main chain) (Å²) | 20.4 | |
| rms deviation in main chain B factor (Å²) | 5.9 | |
| Mean B factor (side chains and waters) (Å²) | 19.7 | |
| rms deviation in side chain B factors (Å²) | 8.2 | |
| Ramachandran plot: | | |
| Residues in most favored region (%) | 87.6 | |
| Residues in additionally allowed region (%) | 11.5 | |
| Residues in generously allowed regions (%) | 0.6 | |
| Residues in disallowed regions (%) | 0.3 | (1 res.) |

^{a}R_{SYM} = ΣΣ|Iᵢ − Iₘ|/ΣΣ Iᵢ where Iᵢ is the intensity of the measured reflection and Iₘ is the mean intensity of all symmetry-related reflections.
^{b}Parameters in parenthesis refer to the outer resolution shell.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are incorporated by reference in their entireties.

What is claimed is:

1. A crystal of a binding complex between β-ketoacyl-ACP synthase I (FabB) and thiolactomycin (TLM) that effectively diffracts X-rays for the determination of the atomic coordinates to a resolution of better than 3.5 Angstroms.

2. The crystal of claim 1, wherein the FabB is E. coli FabB

3. The crystal of claim 2 having space group of P2₁2₁2₁ and a unit cell of dimensions of a=59.1 b=139 and c=211.9 Angstroms.

4. A crystal of a binding complex between β-ketoacyl-ACP synthase I (FabB) and cerulenin that effectively diffracts X-rays for the determination of the atomic coordinates to a resolution of better than 3.5 Angstroms.

5. The crystal of claim 4, wherein the FabB is E. coli FabB.

6. The crystal of claim 5 having space group of P2₁2₁2₁ and a unit cell of dimensions of a=59.2 b=139.6 and c=212.2 Angstroms.

7. A method of obtaining a crystal of an inhibitor-FabB complex comprising growing a crystal of the inhibitor-FabB complex in a buffered solution containing 2.0 M ammonium sulfate, and 20% PEG 400.

8. The method of claim 7 wherein said growing is performed by a method selected from the group consisting of batch crystallization, vapor diffusion, and microdialysis.

9. A computer comprising within its memory a representation of the binding complex between FabB and cerulenin (the FabB-cerulenin binding complex) or a portion of said FabB-cerulenin binding complex, said computer comprising:
(a) a machine-readable data storage medium comprising a data storage material encoded with machine-readable data, wherein said data comprises atomic coordinates from Table III;
(b) a working memory for storing instructions for processing said machine-readable data;
(c) a central processing unit coupled to said working memory and to said machine-readable data storage medium for processing said machine readable data into a three-dimensional representation of the FabB-cerulenin binding complex or a portion of said FabB-cerulenin binding complex; and
(d) a display coupled to said central-processing unit for displaying said three-dimensional representation.

10. A computer comprising within its memory a representation of the binding complex between FabB and thiolactomycin (the FabB-TLM binding complex) or a portion of said FabB-TLM binding complex, said computer comprising:
(a) a machine-readable data storage medium comprising a data storage material encoded with machine-readable data, wherein said data comprises atomic coordinates from Table IV;
(b) a working memory for storing instructions for processing said machine-readable data;
(c) a central processing unit coupled to said working memory and to said machine-readable data storage medium for processing said machine readable data into a three-dimensional representation of the FabB-TLM binding complex or a portion of said FabB-TLM binding complex; and
(d) a display coupled to said central-processing unit for displaying said three-dimensional representation.

11. A method of identifying an agent for use as an inhibitor of bacterial fatty acid synthesis using the crystal of claim 3 comprising:
(a) selecting a potential agent by performing rational drug design with the atomic coordinates determined from the crystal, wherein said selecting is performed in conjunction with computer modeling;
(b) contacting the potential agent with a β-ketoacyl-(Acyl Carrier Protein) synthase; and
(c) measuring the activity of the β-ketoacyl-(Acyl Carrier Protein) synthase;
wherein a potential agent is identified as an agent that inhibits bacterial fatty acid synthesis when there is a decrease in the activity of the β-ketoacyl-(Acyl Carrier Protein) synthase.

12. A method of identifying an agent for use as an inhibitor of bacterial fatty acid synthesis using the crystal of claim 6 comprising:
(a) selecting a potential agent by performing rational drug design with the atomic coordinates determined from the crystal, wherein said selecting is performed in conjunction with computer modeling;
(b) contacting the potential agent with a β-ketoacyl-(Acyl Carrier Protein) synthase; and
(c) measuring the activity of the β-ketoacyl-(Acyl Carrier Protein) synthase; wherein a potential agent is identified as an agent that inhibits bacterial fatty acid synthesis when there is a decrease in the activity of the β-ketoacyl-(Acyl Carrier Protein) synthase.

13. A method of identifying an agent that inhibits bacterial growth using the atomic coordinates obtained from the crystal of claim 3 comprising:
(a) selecting a potential agent by performing rational drug design with the atomic coordinates determined for the crystal, wherein said selecting is performed in conjunction with computer modeling;
(b) contacting the potential agent with a bacterial culture; and
(c) measuring the growth of the bacterial culture; wherein a potential agent is identified as an agent that inhibits bacterial growth when there is a decrease in the growth of the bacterial culture.

14. The method of claim 13, further comprising:
(d) growing a supplemental crystal containing FabB formed in the presence of the potential agent, wherein the crystal effectively diffracts X-rays for the determination of the atomic coordinates to a resolution of better than 5.0 Angstroms;
(e) determining the atomic coordinates of the supplemental crystal with molecular replacement analysis; and
(f) selecting a second generation agent by performing rational drug design with the atomic coordinates determined for the supplemental crystal, wherein said selecting is performed in conjunction with computer modeling.

15. The method of claim 14, further comprising:
(g) contacting the second generation agent with a eukaryotic cell; and
(h) measuring the amount of proliferation of the eukaryotic cell; wherein a potential agent is identified as an agent for inhibiting bacterial growth when there is no change in the proliferation of the eukaryotic cell.

16. A method of identifying an agent that inhibits bacterial growth using the atomic coordinates obtained from the crystal of claim 6 comprising:
(a) selecting a potential agent by performing rational drug design with the atomic coordinates determined for the crystal, wherein said selecting is performed in conjunction with computer modeling;
(b) contacting the potential agent with a bacterial culture; and
(c) measuring the growth of the bacterial culture; wherein a potential agent is identified as an agent that inhibits bacterial growth when there is a decrease in the growth of the bacterial culture.

17. The method of claim 16, further comprising:
(d) growing a supplemental crystal containing FabB formed in the presence of the potential agent, wherein the crystal effectively diffracts X-rays for the determination of the atomic coordinates to a resolution of better than 5.0 Angstroms;
(e) determining the atomic coordinates of the supplemental crystal with molecular replacement analysis; and
(f) selecting a second generation agent by performing rational drug design with the atomic coordinates determined for the supplemental crystal, wherein said selecting is performed in conjunction with computer modeling.

18. The method of claim 17, further comprising:
(g) contacting the second generation agent with a eukaryotic cell; and
(h) measuring the amount of proliferation of the eukaryotic cell; wherein a potential agent is identified as an agent for inhibiting bacterial growth when there is no change in the proliferation of the eukaryotic cell.

19. A method of identifying an agent for use as an inhibitor of bacterial fatty acid synthesis comprising:
(a) selecting a potential agent by performing rational drug design with the set of atomic coordinates in Table III and/or Table IV, wherein said selecting is performed in conjunction with computer modeling;
(b) contacting the potential agent with a bacterial β-ketoacyl-(Acyl Carrier Protein) synthase; and
(c) measuring the activity of the bacterial β-ketoacyl-(Acyl Carrier Protein) synthase; wherein a potential agent is identified as an agent that inhibits bacterial fatty acid synthesis when there is a decrease in the activity of the bacterial β-ketoacyl-(Acyl Carrier Protein) synthase in the presence of the agent relative to in its absence.

20. The method of claim 19, further comprising:
(d) growing a crystal containing a bacterial Fab formed in the presence of the potential agent, wherein the crystal effectively diffracts X-rays for the determination of the atomic coordinates to a resolution of better than 5.0 Angstroms;
(e) determining the atomic coordinates of the crystal with molecular replacement analysis; and
(f) selecting a second generation agent by performing rational drug design with the atomic coordinates determined for the crystal, wherein said selecting is performed in conjunction with computer modeling.

21. A method of identifying an agent that inhibits bacterial growth comprising:
(a) selecting a potential agent by performing rational drug design with the set of atomic coordinates in Table III and/or Table IV, wherein said selecting is performed in conjunction with computer modeling;
(b) contacting the potential agent with a bacterial culture; and
(c) measuring the growth of the bacterial culture; wherein a potential agent is identified as an agent that inhibits bacterial growth when there is a decrease in the growth of the bacterial culture.

22. The method of claim 21, further comprising:
(d) growing a supplemental crystal containing FabB formed in the presence of the potential agent, wherein the crystal effectively diffracts X-rays for the determination of the atomic coordinates to a resolution of better than 5.0 Angstroms;
(e) determining the atomic coordinates of the supplemental crystal with molecular replacement analysis; and
(f) selecting a second generation agent by performing rational drug design with the atomic coordinates determined for the supplemental crystal, wherein said selecting is performed in conjunction with computer modeling.

23. The method of claim 22, further comprising:
(g) contacting the second generation agent with a eukaryotic cell; and
(h) measuring the amount of proliferation of the eukaryotic cell; wherein a potential agent is identified as an agent for inhibiting bacterial growth when there is no change in the proliferation of the eukaryotic cell.

24. A method of selecting a compound that potentially inhibits fatty acid synthesis comprising:
(a) defining the structure of the FabB-inhibitor complex by the atomic coordinates in Table III and/or Table IV; and
(b) selecting a compound which potentially inhibits fatty acid synthesis; wherein said selecting is performed with the aid of the structure defined in step (a).

25. The method of claim 24, further comprising:
(c) contacting the compound with a bacterial β-ketoacyl-(Acyl Carrier Protein) synthase; and
(d) measuring the activity of the bacterial β-ketoacyl-(Acyl Carrier Protein) synthase; wherein the compound is identified as an agent that inhibits bacterial β-ketoacyl-(Acyl Carrier Protein) synthase when there is a decrease in the activity of the bacterial β-ketoacyl-(Acyl Carrier Protein) synthase in the presence of the compound relative to in its absence.


SEQUENCE LISTING (4 sequences)

SEQ ID NO: 1 - protein, 403 aa, Escherichia coli
KRAVITGLGIVSSIGNNQQEVLASLREGRSGITFSQELKDSGMRSHVWGNVKLDTTGLIDRKVVRFMSDASIYAFLSMEQAIADAGLSPEAYQNNPRVGLIAGSGGGSPRFQVFGADAMRGPRGLKAVGPYVVTKAMASGVSACLATPFKIHGVNYSISSACATSAHCIGNAVEQIQLGKQDIVFAGGGEELCWEMACEFDAMGALSTKYNDTPEKASRTYDAHRDGFVIAGGGGMVVVEELEHALARGAHIYAEIVGYGATSDGADMVAPSGEGAVRCMKMAMHGVDTPIDYLNSHGTSTPVGDVKELAAIREVFGDKSPAISATKAMTGHSLGAAGVQEAIYSLLMLEHGFIAPSINIEELDEQAAGLNIVTETTDRELTTVMSNSFGFGGTNATLVMRKL

SEQ ID NO: 2 - protein, 406 aa, Escherichia coli
MKRAVITGLGIVSSIGNNQQEVLASLREGRSGITFSQELKDSGMRSHVWGNVKLDTTGLIDRKVVRFMSDASIYAFLSMEQAIADAGLSPEAYQNNPRVGLIAGSGGGSPRFQVFGADAMRGPRGLKAVGPYVVTKAMASGVSACLATPFKIHGVNYSISSACATSAHCIGNAVEQIQLGKQDIVFAGGGEELCWEMACEFDAMGALSTKYNDTPEKASRTYDAHRDGFVIAGGGGMVVVEELEHALARGAHIYAEIVGYGATSDGADMVAPSGEGAVRCMKMAMHGVDTPIDYLNSHGTSTPVGDVKELAAIREVFGDKSPAISATKAMTGHSLGAAGVQEAIYSLLMLEHGFIAPSINIEELDEQAAGLNIVTETTDRELTTVMSNSFGFGGTNATLVMRKLKD

SEQ ID NO: 3 - DNA, 22 bp, Artificial Sequence
  primer
aaagccatgaccggtaactctc

SEQ ID NO: 4 - DNA, 26 bp, Artificial Sequence
  primer
gcaggatccggcgattgtcaatgatg